(19) United States
(12) Patent Application Publication
(10) Pub. No.: US 2006/0287313 A1
(43) Pub. Date: Dec. 21, 2006

(54) Isoquinoline compounds and methods of use thereof

(75) Inventors: Prakash Jagtap, North Andover, MA (US); Csaba Szabo, Budapest (HU)

Correspondence Address: WILMER CUTLER PICKERING HALE AND DORR LLP, 399 PARK AVENUE, NEW YORK, NY, 10022, US

(73) Assignee: Inotek Pharmaceuticals Corporation, Beverly, MA (US)

(21) Appl. No.: 11/355,285

(22) Filed: Feb. 15, 2006

Related U.S. Application Data
(60) Provisional application No. 60/656,638, filed on Feb. 25, 2005.

(51) Int. Cl. A61K 31/5377; A61K 31/496; A61K 31/473; C07D 413/02; C07D 403/02; C07D 221/18

(52) U.S. Cl. 514/232.5; 544/125; 544/361; 546/71; 514/253.02; 514/284

(57) ABSTRACT

The present invention relates to Isoquinoline Compounds, compositions comprising an effective amount of an Isoquinoline Compound and methods for treating or preventing an inflammatory disease, a reperfusion injury, diabetes, a diabetic complication, reoxygenation injury resulting from organ transplantation, an ischemic condition, Parkinson's disease, renal failure, a vascular disease, or cancer, comprising administering to a subject in need thereof an effective amount of an Isoquinoline Compound.

This application claims the benefit of U.S. Provisional application Ser. No. 60/656,638, filed Feb. 25, 2005, which is currently pending, the entirety of which is incorporated herein by reference.

### 1. FIELD OF THE INVENTION

The present invention relates to Isoquinoline Compounds, compositions comprising an effective amount of an Isoquinoline Compound and methods for treating or preventing an inflammatory disease, a reperfusion injury, diabetes, a diabetic complication, reoxygenation injury resulting from organ transplantation, an ischemic condition, Parkinson's disease, renal failure, a vascular disease, or cancer, comprising administering to a subject in need thereof an effective amount of an Isoquinoline Compound.

### 2. BACKGROUND OF THE INVENTION

Inflammatory diseases, such as arthritis, colitis, and autoimmune diabetes, typically manifest themselves as disorders distinct from those associated with reperfusion injuries, e.g., stroke and heart attack, and can clinically manifest themselves as different entities. However, there can be common underlying mechanisms between these two types of disorders. In particular, inflammatory disease and reperfusion injury can induce proinflammatory cytokine and chemokine synthesis which can, in turn, result in production of cytotoxic free radicals such as nitric oxide and superoxide. NO and superoxide can react to form peroxynitrite (ONOO⁻) (Szabó et al., Shock 6:79-88, 1996).

The ONOO⁻-induced cell necrosis observed in inflammatory disease and in reperfusion injury involves the activation of the nuclear enzyme poly(ADP-ribose) synthetase (PARS). Activation of PARS is thought to be an important step in the cell-mediated death observed in inflammation and reperfusion injury (Szabó et al., Trends Pharmacol. Sci. 19:287-98, 1998).

A number of PARS inhibitors have been described in the art. See, e.g., Banasik et al., J. Biol. Chem., 267:1569-75, 1992, and Banasik et al., Mol. Cell. Biochem., 138:185-97, 1994; WO 00/39104; WO 00/39070; WO 99/59975; WO 99/59973; WO 99/11649; WO 99/11645; WO 99/11644; WO 99/11628; WO 99/11623; WO 99/11311; WO 00/42040; Zhang et al., Biochem. Biophys. Res. Commun., 278:590-98, 2000; White et al., J. Med. Chem., 43:4084-4097, 2000; Griffin et al., J. Med. Chem., 41:5247-5256, 1998; Shinkwin et al., Bioorg. Med. Chem., 7:297-308, 1999; and Soriano et at., Nature Medicine, 7:108-113, 2001. Adverse effects associated with administration of PARS inhibitors have been discussed in Milan et al., Science, 223:589-591, 1984.

Isoquinoline compounds have been previously discussed in the art. For example, cytotoxic non-camptothecin topoisomerase I inhibitors are reported in Cushman et al., J. Med. Chem., 43:3688-3698, 2300 and Cushman et al., J. Med. Chem. 42:446-57, 1999; indeno[1,2-c]isoquinolines are reported as antineoplastic agents in Cushman et al., WO 00/21537; and as neoplasm inhibitors in Hrbata et al., WO 93/05023.

Syntheses of isoquinoline compounds have been reported. For example, see Wawzonek et al., Org. Prep. Proc. Int., 14:163-8, 1982; Wawzonek et al., Can. J. Chem., 59:2833, 1981; Andoi et al., Bull. Chem. Soc. Japan, 47:1014-17, 1974; Dusemund et al., Arch. Pharm (Weinheim, Ger.), 3 17:381-2, 1984; and Lal et al., Indian J. Chem., Sect. B, 38B:33-39, 1999.

There remains, however, a need in the art for compounds useful for treating or preventing an inflammatory disease, a reperfusion injury, diabetes, a diabetic complication, reoxygenation injury resulting from organ transplantation, an ischemic condition, Parkinson's disease, renal failure, a vascular disease, or cancer.

Citation of any reference in Section 2 of this application is not an admission that the reference is prior art.

### 3. SUMMARY OF THE INVENTION

In one aspect the invention provides a compound of Formula (I) 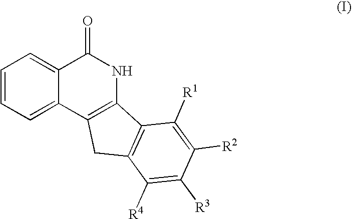
and pharmaceutically acceptable salts thereof;
wherein

R² and R³ are hydrogen;

one of the R¹ and R⁴ groups is —NHC(O)—(CH₂)ₙ—NR⁵R⁶ and the remaining group is hydrogen;

R⁵ and R⁶ are independently —H, —C₁-C₆ alkyl, -phenyl, or benzyl, wherein the —C₁-C₆ alkyl, -phenyl, or benzyl, is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; and

n is an integer ranging from 1 to 6.

In another aspect the invention includes a compound of Formula (II) 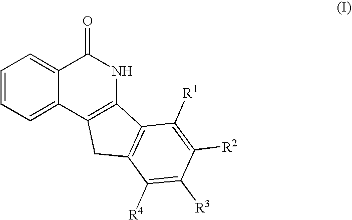
and pharmaceutically acceptable salts thereof
wherein

one of the R¹, R², R³, and R⁴ groups is —NHC(O)—(CH₂)ₙ—NZ₁Z₂ and the remaining groups are simultaneously hydrogen;

one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₁ and Z₂ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; and

n is an integer ranging from 1 to 6.

In another aspect the invention provides compounds of the formula (III): 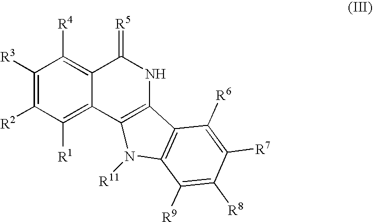
and pharmaceutically acceptable salts thereof,
wherein:

R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂-halo, —OH, —NH₂, or -A-B;

R⁵ is O, S or NH;

A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;

B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, (nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, -C₁-C₁₀ alkyl, -aryl, —C(O)OH, or —C(O)O—(C₁-C₅ alkyl);

Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);

R¹¹ is —C(O)O—(C₁-C₅ alkylene)-NZ₅Z₆;

one of Z₅ and Z₆ is —H, —C₁-C₆ alkyl or -phenyl, and the other of Z₅ and Z₆ is phenyl, wherein the -phenyl in each instance is unsubstituted or substituted with one or more of -halo, —OH or —N(Z₇)(Z₈), where N, Z₇ and Z₈ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₅ and Z₆ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl;

each n is independently an integer ranging from 1 to 10; and

each p is independently an integer ranging from 0 to 5.

The present invention further encompasses compounds having the Formula (IV): 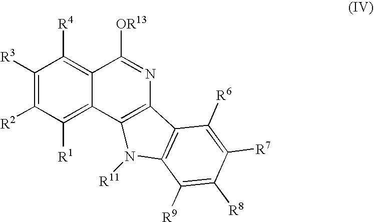
and pharmaceutically acceptable salts thereof,
wherein:

R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂, -halo, —OH, —NH₂, or -A-B;

A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;

B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, (nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);

Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);

R¹¹ is —C(O)O—(C₁-C₅ alkylene)-NZ₅Z₆;

one of Z₅ and Z₆ is —H, —C₁-C₆ alkyl, or -phenyl, and the other of Z₅ and Z₆ is phenyl, wherein the -phenyl in each instance is unsubstituted or substituted with one or more of -halo, —OH or —N(Z₇)(Z₈), where N, Z₇ and Z₈ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₅ and Z₆ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl;

R¹³ is —C₁-C₁₀ alkyl, —C(O)—C₁-C₁₀ alkyl, —C(O)-aryl, —C(O)-(3- to 7-membered monocyclic heterocycle), or -glycoside, each of which is unsubstituted or substituted with one or more -halo, —C(O)OH, or —OH groups;

each n is independently an integer ranging from 1 to 10; and

each p is independently an integer ranging from 0 to 5.

The present invention further encompasses compounds of Formula (V) 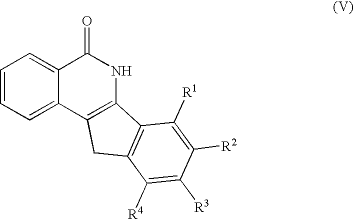
and pharmaceutically acceptable salts thereof
wherein

R³ is —NHC(O)—(CH₂)ₙ—X and R₁, R₂, R₄ are simultaneously hydrogen;

X is —OH, hydroxy-substituted C₁-C₆ alkyl, or NZ₁Z₂;

one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₁ and Z₂ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; and

n is 0 or 1.

A compound of Formula (I), Formula (II), Formula (III), Formula (IV) or Formula (V), or a pharmaceutically acceptable salt thereof (each being an “Isoquinoline Compound”) is useful for treating or preventing an inflammatory disease, a reperfusion injury, diabetes, a diabetic complication, reoxygenation injury resulting from organ transplantation, an ischemic condition, Parkinson's disease, renal failure, a vascular disease, or cancer (each being a “Condition”).

Also provided by the invention are methods for treating or preventing a Condition, comprising administering to a subject in need of such treatment or prevention an effective amount of an Isoquinoline Compound.

The invention further provides compositions comprising and an effective amount of an Isoquinoline Compound and a physiologically acceptable carrier or vehicle.

The details of the invention are set forth in the accompanying description below.

Although any methods and materials similar or equivalent to those described herein can be used in the practice or testing of the present invention, illustrative methods and materials are now described. Other features, objects, and advantages of the invention will be apparent from the description and from the claims. All patents and publications cited in this specification are incorporated by reference.

### 4. DETAILED DESCRIPTION OF THE INVENTION

### 4.1 Definitions and Abbreviations

The following definitions are used in connection with the Isoquinoline Compounds:

The term “—C₁-C₅ alkyl” as used herein, refers to a straight chain or branched non-cyclic hydrocarbon having from 1 to 5 carbon atoms, wherein one of the hydrocarbon's hydrogen atoms has been replaced by a single bond. Representative straight chain —C₁-C₅ alkyls include -methyl, -ethyl, -n-propyl, -n-butyl and -n-pentyl. Representative branched —C₁-C₅ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

The term “—C₁-C₆ alkyl” as used herein, refers to a straight chain or branched non-cyclic hydrocarbon having from 1 to 6 carbon atoms, wherein one of the hydrocarbon's hydrogen atoms has been replaced by a single bond. Representative straight chain —C₁-C₆ alkyls include -methyl, -ethyl, -n-propyl, -n-butyl, -n-pentyl and -n-hexyl. Representative branched —C₁-C₆ alkyls include -isopropyl, -sec-butyl, -isobutyl, -tert-butyl, -isopentyl, -neopentyl, -1-methylbutyl, -isohexyl, -neohexyl, -2-methylbutyl, -3-methylbutyl, -1,1-dimethylpropyl and -1,2-dimethylpropyl.

The term “—C₁-C₁₀ alkyl” as used herein, refers to a straight chain or branched non-cyclic hydrocarbon having from 1 to 10 carbon atoms, wherein one of the hydrocarbon's hydrogen atoms has been replaced by a single bond. Representative —C₁-C₁₀ alkyls include methyl, ethyl, propyl, butyl, pentyl, hexyl, heptyl, octyl, -nonyl, decyl, isopropyl, isobutyl, sec-butyl and tert-butyl, isopentyl, neopentyl, isohexyl, isoheptyl, isooctyl, isononyl and isodecyl.

The term “—(C₂-C₁₀)alkenyl” as used herein, refers to a straight chain or branched non-cyclic hydrocarbon having from 2 to 10 carbon atoms and including at least one carbon-carbon double bond. Representative straight chain and branched (C₂₋C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “—(C₂-C₁₀)alkynyl” as used herein, refers to a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

The term “—C₁-C₅ alkylene-” as used herein, refers to a straight chain or branched acyclic hydrocarbon having from 1-5 carbon atoms, wherein two of the hydrocarbon's hydrogen atoms has been replaced by a single bond. Representative —C₁-C₅-alkylene groups include methylene, ethylene, propylene, butylene, and pentylene. Other —C₁-C₅-alkylene groups include —CH(CH₃)—, —CH₂CH(CH₃)—, —CH₂CH₂CH(CH₃)—, —CH₂CH(CH₃)CH₂—, —CH(CH₃)CH(CH₃)—, —CH₂CH₂CH₂CH(CH₃)—, —CH₂CH₂CH(CH₃)CH₂—, —CH(CH₃)CH(CH₃)CH₂— and —CH(CH₃)CH₂CH(CH₃)—.

The term “—(C₃-C₈)monocyclic cycloalkyl” as used herein, refers to a saturated cyclic hydrocarbon having from 3 to 8 carbon atoms. Representative (C₃-C₈)cycloalkyls include -cyclopropyl, -cyclobutyl, -cyclopentyl, -cyclohexyl, -cycloheptyl and -cyclooctyl.

The term “—(C₈-C₁₄) bicyclic cycloalkyl” as used herein, refers to a bi-cyclic hydrocarbon ring system having from 8 to 14 carbon atoms and at least one saturated cyclic alkyl ring. Representative —(C₈-C₁₄) bicycloalkyls include -indanyl, -1,2,3,4-tetrahydronaphthyl, -5,6,7,8-tetrahydronaphthyl, -perhydronaphthyl and the like.

The term “—(C₅-C₈)monocyclic cycloalkenyl” as used herein, refers to a cyclic non-aromatic hydrocarbon having at least one carbon-carbon double bond in the cyclic system and from 5 to 8 carbon atoms. Representative (C₄-C₈)monocyclic cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl and the like.

The term “—(C₈-C₁₄) bicyclic cycloalkenyl” as used herein, refers to a bi-cyclic hydrocarbon ring system having at least one carbon-carbon double bond in each ring and from 8 to 14 carbon atoms. Representative —(C₈-C₁₄) bicyclic cycloalkenyls include -indenyl, -pentalenyl, -naphthalenyl, -azulenyl, -heptalenyl, -1,2,7,8-tetrahydronaphthalenyl and the like.

A “nitrogen containing 3- to 7-membered monocyclic heterocycle” refers to a monocyclic 3- to 7-membered aromatic or non-aromatic monocyclic cycloalkyl group in which one of the cycloalkyl group's ring carbon atoms has been replaced with a nitrogen atom and 0-4 of the cycloalkyl group's remaining ring carbon atoms may be independently replaced with a N, O or S atom. The nitrogen containing 3- to 7-membered monocyclic heterocycles can be attached via a nitrogen, sulfur, or carbon atom. Representative examples of nitrogen-containing-3- to 7-membered monocyclic heterocycles include, but are not limited to, piperidinyl, piperazinyl, pyrrolyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, morpholinium, and morpholinyl. In one embodiment, a nitrogen containing 3- to 7-membered monocyclic heterocycle is substituted with up to three groups, independently chosen from: —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

The term “glycoside” as used herein refers to a hexose or a pentose sugar forming an α- or β-glycosidic linkage. Representative examples of glycosides include, but are not limited to ribose, deoxyribose, fructose, galactose, glucuronic acid and glucose.

“Halo” is —F, —Cl, —Br or —I.

A “subject” is a mammal, e.g., a human, mouse, rat, guinea pig, dog, cat, horse, cow, pig, or non-human primate, such as a monkey, chimpanzee, baboon or rhesus. In one embodiment, the subject is a human.

Representative “pharmaceutically acceptable salts” include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A hydrate is another example of a pharmaceutically acceptable salt.

In one embodiment, the pharmaceutically acceptable salt is a mesylate salt.

In another embodiment, the pharmaceutically acceptable salt is a camphorsulfonate salt.

An “effective amount” when used in connection with an Isoquinoline Compound is an amount that is effective for treating or preventing a Condition.

An “effective amount” when used in connection with another anticancer agent is an amount that is effective for treating or preventing cancer alone or in combination with an Isoquinoline Compound. “In combination with” includes administration within the same composition and within separate compositions. In the latter instance, the anticancer agent is administered during a time when the Isoquinoline Compound exerts its prophylactic or therapeutic effect, or vice versa.

The following abbreviations used herein have the following meanings: AIBN is azibisisobutyronitrile, DIEA is diisopropylethylamine, DMF is dimethyl formamide, DMSO is dimethyl sulfoxide, DPPA is diphenylphosphorylazide, Et₃N is triethylamine, EtOH is ethanol, MeCN is acetonitrile, MeOH is methanol, NaH is sodium hydride, NBS is N-bromosuccinimide, PPA is polyphosphoric acid, py is pyridine, THF is tetrahydrofuran, and TMZ is temozolomide.

### 4.2 The Isoquinoline Compounds of Formula (I)

The present invention provides Isoquinoline Compounds according to Formula (I), below: 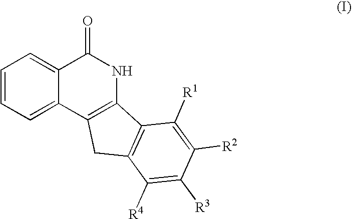
and pharmaceutically acceptable salts thereof,
wherein:

R¹, R², R³ and R⁴ are as defined above for the compounds of formula (I).

In one embodiment, R¹ is —NH(CH₂)ₙ—N(R⁵)(R⁶) and R², R³ and R⁴ are each hydrogen.

In still another embodiment, R⁴ is —NH(CH₂)ₙ—N(R⁵)(R⁶) and R¹, R² and R³ are each hydrogen.

In one embodiment, R⁵ and R⁶ are each C₁-C₆ alkyl.

In another embodiment, R⁵ and R⁶ are each methyl.

In one embodiment, n is 1.

In another embodiment, n is 2.

In still another embodiment, n is 3.

In yet another embodiment, n is 4.

In a further embodiment, n is 5.

In another embodiment N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

In various embodiments, —N(R⁵)(R⁶) is:

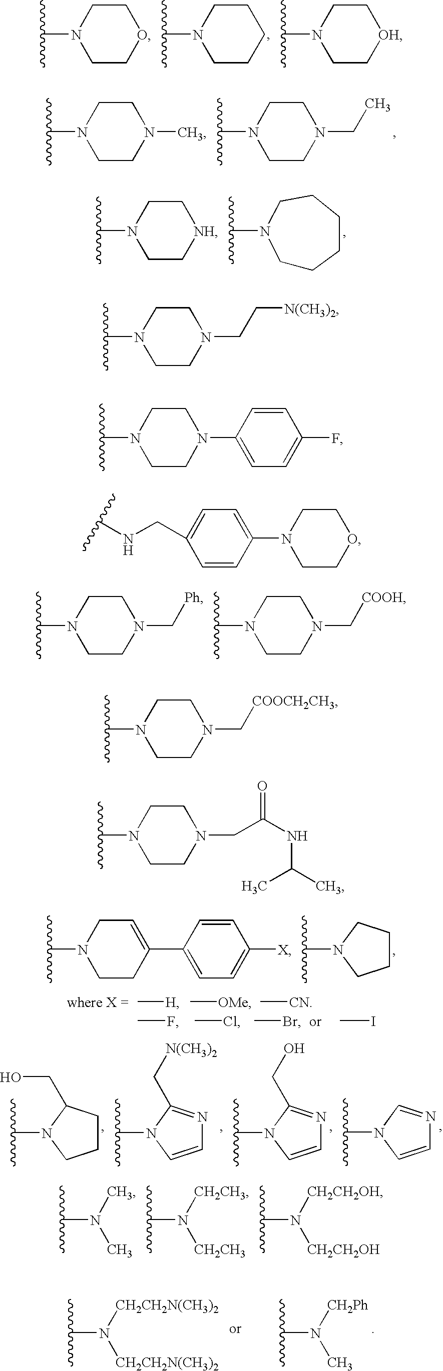

Illustrative examples of the Isoquinoline Compounds of Formula (I) include the compounds of Formula (Ia) as set forth below:

| | |
|---|---|
| | (Ia) |
| 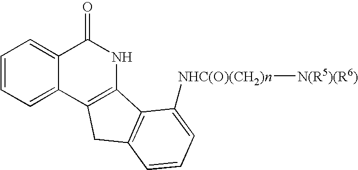 | |

| Compound | n | —N(R⁵)(R⁶) |
|---|---|---|
| 1a | 1 | —N(CH₃)₂ |
| 1b | 2 | —N(CH₃)₂ |
| 1c | 3 | —N(CH₃)₂ |
| 1d | 4 | —N(CH₃)₂ |
| 1e | 5 | —N(CH₃)₂ |
| 2a | 1 | 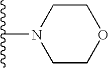 |
| 2b | 2 | 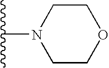 |
| 2c | 3 | 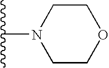 |
| 2d | 4 | 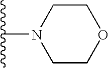 |
| 2e | 5 | 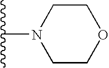 |

and pharmaceutically acceptable salts thereof.

Other illustrative examples of the Isoquinoline Compounds of Formula (I) include the compounds of Formula (Ib) as set forth below:

| | |
|---|---|
| | (Ib) |
| 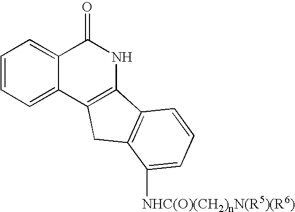 | |

| Compound | n | —N(R⁵)(R⁶) |
|---|---|---|
| 3a | 1 | —N(CH₃)₂ |
| 3b | 2 | —N(CH₃)₂ |
| 3c | 3 | —N(CH₃)₂ |
| 3d | 4 | —N(CH₃)₂ |
| 3e | 5 | —N(CH₃)₂ |
| 4a | 1 | 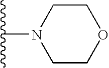 |
| 4b | 2 | 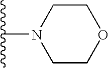 |
| 4c | 3 | 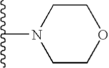 |
| 4d | 4 | 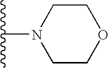 |
| 4e | 5 | 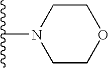 |

and pharmaceutically acceptable salts thereof.

### 4.3 The Isoquinoline Compounds of Formula (II)

The present invention provides Isoquinoline Compounds according to Formula (II), below: 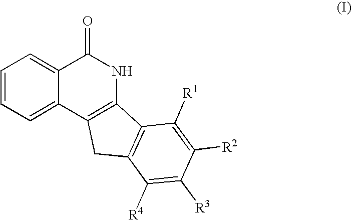
and pharmaceutically acceptable salts thereof,
wherein:

R¹, R², R³ and R⁴ are as defined above for the compounds of formula (II).

In one embodiment, R¹ is —NHC(O)—(CH₂)ₙ—N(Z₁)(Z₂) and R², R³ and R⁴ are each hydrogen.

In another embodiment, R² is —NHC(O)—(CH₂)ₙ—N(Z₁)(Z₂) and R¹, R³ and R⁴ are each hydrogen.

In another embodiment, R³ is —NHC(O)—(CH₂)ₙ—N(Z₁)(Z₂) and R¹, R² and R⁴ are each hydrogen.

In still another embodiment, R⁴ is —NHC(O)—(CH₂)ₙ—N(Z₁)(Z₂) and R¹, R² and R³ are each hydrogen.

In one embodiment, n is 1.

In another embodiment, n is 2.

In still another embodiment, n is 3.

In yet another embodiment, n is 4.

In a further embodiment, n is 5.

In another embodiment N, Z₁ and Z₂ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

In various embodiments, —N(Z₁)(Z₂) is:

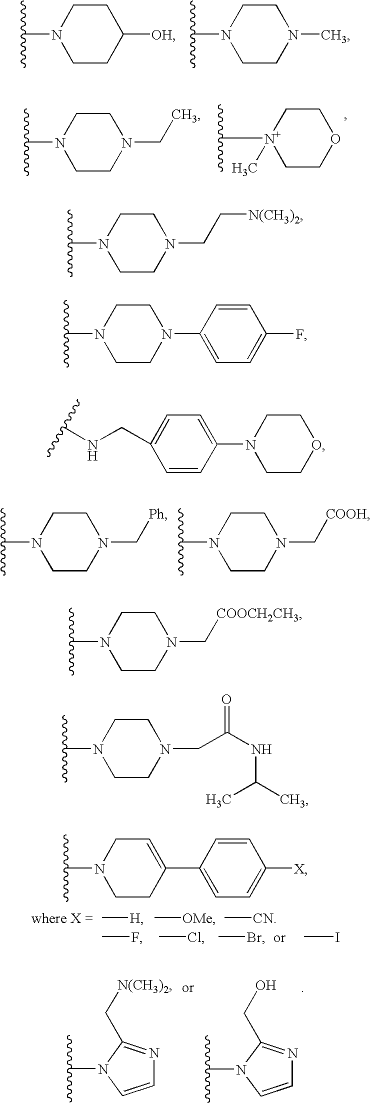

Illustrative examples of the Isoquinoline Compounds of Formula (II) include the compounds of Formula (IIa) as set forth below:

| | |
|---|---|
| | (IIa) |
| 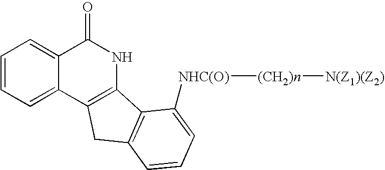 | |

| Compound | n | —N(Z₁)(Z₂) |
|---|---|---|
| 5a | 1 | 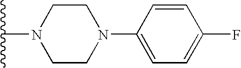 |
| 5b | 2 | 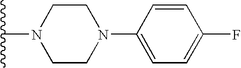 |
| 5c | 3 | 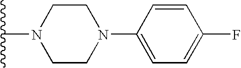 |
| 5d | 4 | 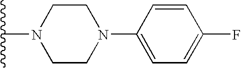 |
| 5e | 5 | 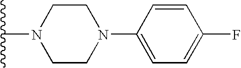 |
| 6a | 1 | 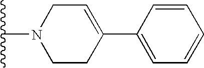 |
| 6b | 2 | 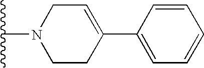 |
| 6c | 3 | 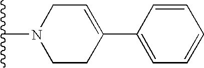 |
| 6d | 4 | 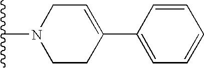 |
| 6e | 5 | 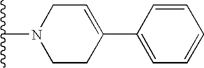 |
| 7a | 1 | 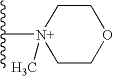 |
| 7b | 2 | 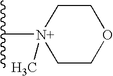 |
| 7c | 3 | 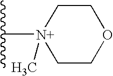 |
| 7d | 4 | 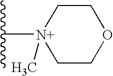 |
| 7e | 5 | 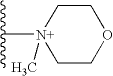 |

and pharmaceutically acceptable salts thereof.

Other illustrative examples of the Isoquinoline Compounds of Formula (II) include the compounds of Formula (IIb) as set forth below:

| | |
|---|---|
| | (IIb) |
| 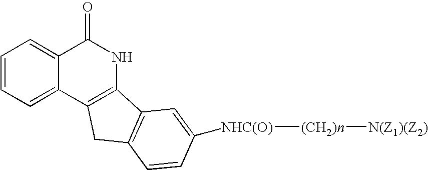 | |

| Compound | n | —N(Z₁)(Z₂) |
|---|---|---|
| 8a | 1 | 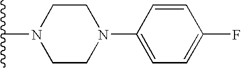 |
| 8b | 2 | 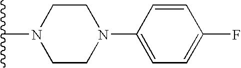 |
| 8c | 3 | 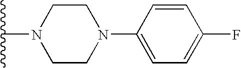 |
| 8d | 4 | 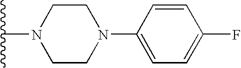 |
| 8e | 5 | 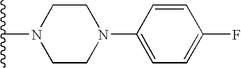 |
| 9a | 1 | 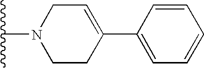 |
| 9b | 2 | 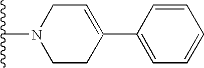 |
| 9c | 3 | 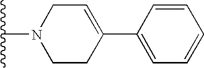 |
| 9d | 4 | 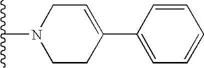 |
| 9e | 5 | 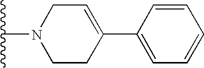 |
| 10a | 1 | 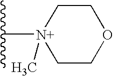 |
| 10b | 2 | 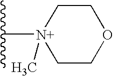 |
| 10c | 3 | 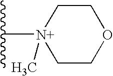 |
| 10d | 4 | 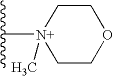 |
| 10e | 5 | 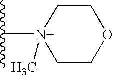 |

and pharmaceutically acceptable salts thereof.

Other illustrative examples of the Isoquinoline Compounds of Formula (II) include the compounds of Formula (IIc) as set forth below:

| | |
|---|---|
| | (IIc) |
| 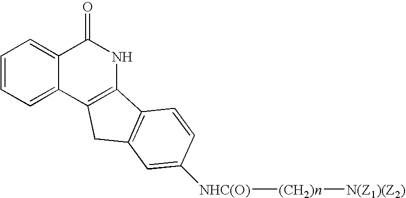 | |

| Compound | n | —N(Z₁)(Z₂) |
|---|---|---|
| 11a | 1 | 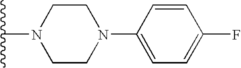 |
| 11b | 2 | 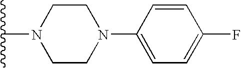 |
| 11c | 3 | 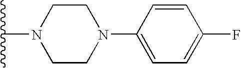 |
| 11d | 4 | 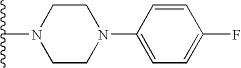 |
| 11e | 5 | 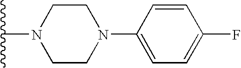 |
| 12a | 1 | 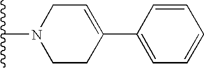 |
| 12b | 2 | 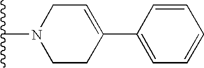 |
| 12c | 3 | 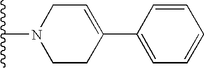 |
| 12d | 4 | 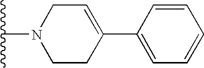 |
| 12e | 5 | 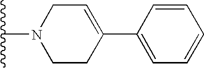 |
| 13a | 1 | 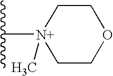 |
| 13b | 2 | 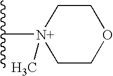 |
| 13c | 3 | 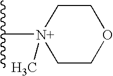 |
| 13d | 4 | 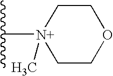 |
| 13e | 5 | 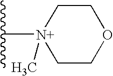 |
| 24a | 1 | 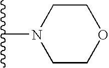 |
| 25 | 1 | 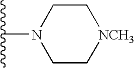 |
| 26 | 1 | 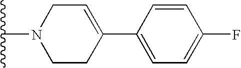 |

and pharmaceutically acceptable salts thereof.

Other illustrative examples of the Isoquinoline Compounds of Formula (II) include the compounds of Formula (IId) as set forth below:

| | |
|---|---|
| | (IId) |
| 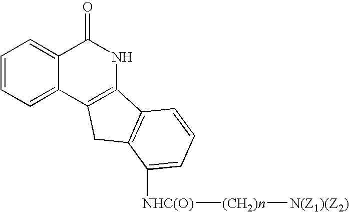 | |

| Compound | n | —N(Z₁)(Z₂) |
|---|---|---|
| 14a | 1 | 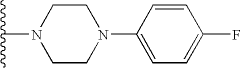 |
| 14b | 2 | 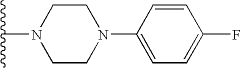 |
| 14c | 3 | 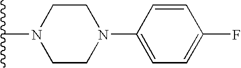 |
| 14d | 4 | 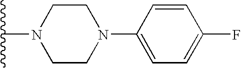 |
| 14e | 5 | 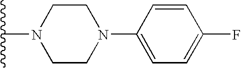 |
| 15a | 1 | 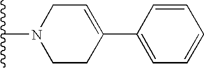 |
| 15b | 2 | 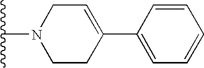 |
| 15c | 3 | 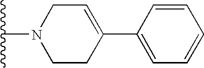 |
| 15d | 4 | 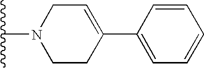 |
| 15e | 5 | 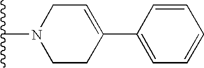 |
| 16a | 1 | 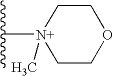 |
| 16b | 2 | 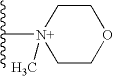 |
| 16c | 3 | 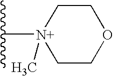 |
| 16d | 4 | 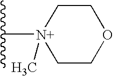 |
| 16e | 5 | 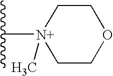 |

and pharmaceutically acceptable salts thereof.

### 4.4 The Isoquinoline Compounds of Formula (III)

The present invention provides Isoquinoline Compounds according to Formula (III), below: 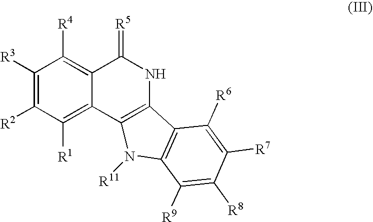
where R¹, R², R³, R⁴, R⁵, R⁶, R⁷, R⁸, R⁹, and R¹¹ are defined above for the Isoquinoline Compounds of Formula (III).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In yet another embodiment R², R³ and R⁴ are each —H.

In another embodiment R⁶ and R⁹ are each —H.

In another embodiment R⁶, R⁷, R⁸ and R⁹ are each —H.

In still another embodiment R¹, R², R³, R⁴, R⁶, R⁷, R⁸ and R⁹ are each —H.

In one embodiment R⁵ is O.

In another embodiment, R⁵ is S.

In yet another embodiment, R⁵ is NH.

In another embodiment R⁷ is —H and R⁸ is -A-B, where A is —NHC(O)— and B is —C₁-C₅ alkylene)-NZ₁Z₂.

In still another embodiment R⁸ is —H and R⁷ is -A-B, where A is —NHC(O)— and B is C₁-C₅ alkylene)-NZ₁Z₂.

In yet another embodiment R⁷ is —H and R⁸ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In a further embodiment R⁸ is —H and R⁷ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In another embodiment R⁷ is —H and R⁸ is —NHC(O)CH₂N(CH₃)₂.

In another embodiment R⁷ is —H and R⁸ is —SO₂NH(CH₂)₃—(N-morpholino).

In a further embodiment R⁸ is —H and R⁷ is —SO₂NH(CH₂)₃—(N-morpholino).

In a further embodiment R¹-R⁴ are each —H, R⁵ is O, and R¹¹ is or —C(O)O—(C₁-C₅ alkyl)-NZ₁Z₂.

In another embodiment N, Z₅ and Z₆ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

In various embodiments, R¹¹ is —C(O)O—(C₁-C₅ alkylene)-NZ₅Z₆, where —N(Z₅)(Z₆) is:

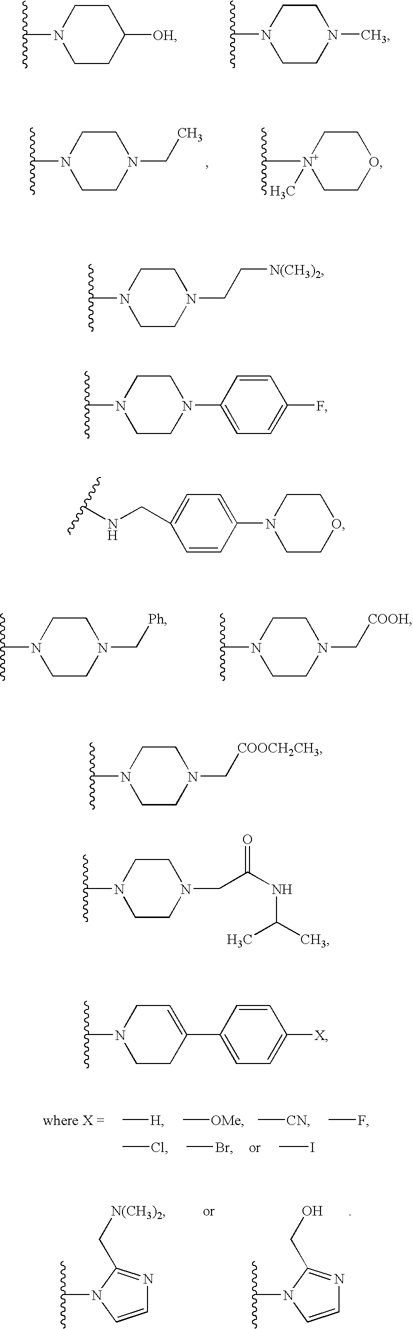

Other illustrative examples of the Isoquinoline Compounds of Formula (III) include the compounds of Formula (IIIa) as set forth below:

| | |
|---|---|
| | (IIIa) |
| 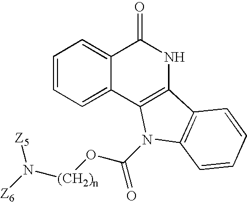 | |

| Compound | n | —N(Z₅)(Z₆) |
|---|---|---|
| 17a | 1 | 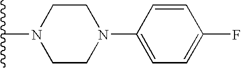 |
| 17b | 2 | 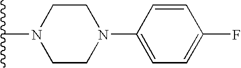 |
| 17c | 3 | 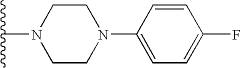 |
| 17d | 4 | 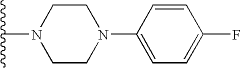 |
| 17e | 5 | 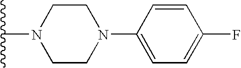 |
| 18a | 1 | 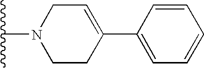 |
| 18b | 2 | 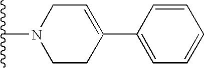 |
| 18c | 3 | 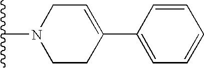 |
| 18d | 4 | 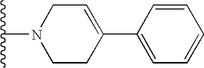 |
| 18e | 5 | 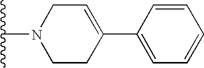 |
| 19a | 1 | 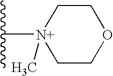 |
| 19b | 2 | 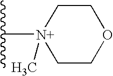 |
| 19c | 3 | 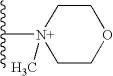 |
| 19d | 4 | 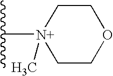 |
| 19e | 5 | 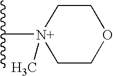 |

and pharmaceutically acceptable salts thereof.

### 4.5 The Isoquinoline Compounds of Formula (IV)

The present invention provides Isoquinoline Compounds according to Formula (IV), below: 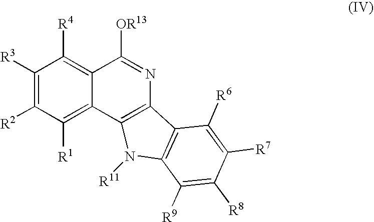
where R¹, R², R³, R¹, R⁶, R⁷, R⁸, R⁹, R¹¹, and R¹³ are defined above for the Isoquinoline Compounds of Formula (IV).

In one embodiment R¹, R², R³ and R⁴ are independently —H, —F, —NO₂, —NH₂, —OH, or —O—(C₁-C₅ alkyl).

In another embodiment R¹, R², R³ and R⁴ are each —H.

In yet another embodiment R², R³ and R⁴ are each —H.

In another embodiment R⁶ and R⁹ are each —H.

In another embodiment R⁶, R⁷, R⁸ and R⁹ are each —H.

In still another embodiment R¹, R², R³, R⁴, R⁶, R⁷, R⁸ and R⁹ are each —H.

In another embodiment R⁷ is —H and R⁸ is -A-B, where A is —NHC(O)— and B is —(C₁-C₅ alkylene)-NZ₁Z₂.

In still another embodiment R⁸ is —H and R⁷ is -A-B, where A is —NHC(O)— and B is C₁-C₅ alkylene)-NZ₁Z₂.

In yet another embodiment R⁷ is —H and R⁸ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In a further embodiment R⁸ is —H and R⁷ is -A-B, where A is —SO₂NH—; B is —C₁-C₅ alkylene)-N(Z₁)(Z₂); and N, Z₁ and Z₂ are taken together to form a nitrogen-containing 3- to 7-membered monocyclic heterocycle.

In another embodiment R⁷ is —H and R⁸ is —NHC(O)CH₂N(CH₃)₂.

In another embodiment R⁷ is —H and R⁸ is —SO₂NH(CH₂)₃—(N-morpholino).

In a further embodiment R⁸ is —H and R⁷ is —SO₂NH(CH₂)₃—(N-morpholino).

In another embodiment, the compounds of Formula (IV) are those wherein R¹, R⁷ and R⁸ are —H.

In another embodiment N, Z₅ and Z₆ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

In various embodiments, R¹¹ is —C(O)O—(C₁-C₅ alkylene)-NZ₅Z₆, where —N(Z₅)(Z₆) is:

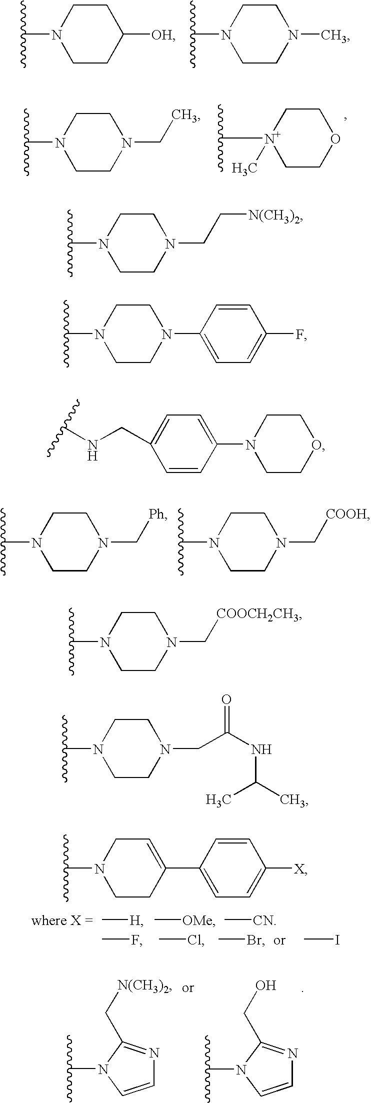

Other illustrative examples of the Isoquinoline Compounds of Formula (IV) include the compounds of Formula (IVa) as set forth below: 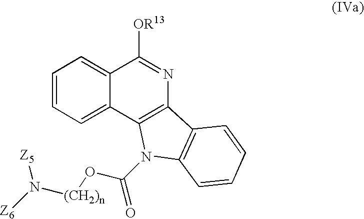
and pharmaceutically acceptable salts thereof,
wherein R³, n, Z₅, and Z₆ are as set forth above for compounds of Formula IV.

### 4.6 The Isoquinoline Compounds of Formula (V)

The present invention provides Isoquinoline Compounds according to Formula (V), below: 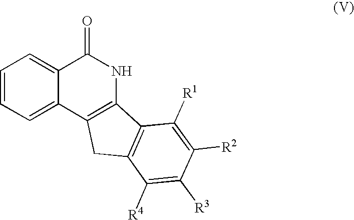
and pharmaceutically acceptable salts thereof,
wherein:

R¹, R², R³ and R⁴ are as defined above for the compounds of formula (V).

In one embodiment, R³ is —NHC(O)—(CH₂)ₙ—N(Z₁)(Z₂) and R¹, R² and R⁴ are each hydrogen.

In another embodiment, R³ is —NHC(O)—(CH₂)ₙ—OH and R¹, R² and R⁴ are each hydrogen.

In one embodiment, n is 1.

In another embodiment, n is 0.

In still another embodiment, Z₁ is H and Z₂ is propyl.
Other illustrative examples of the Isoquinoline Compounds of Formula (V) include the compounds 59 and 60 as set forth below:

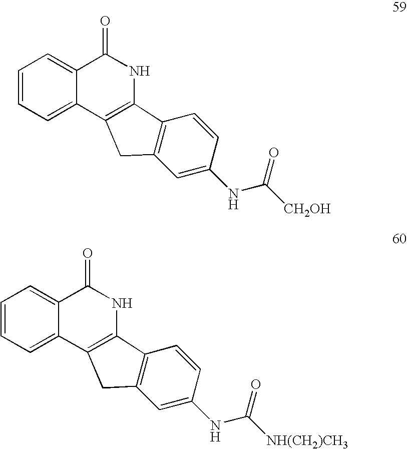

### 4.7 Methods for Making the Isoquinoline Compounds

Methods useful for making the Isoquinoline Compounds are set forth in the Examples below and generalized in Schemes 1-2.

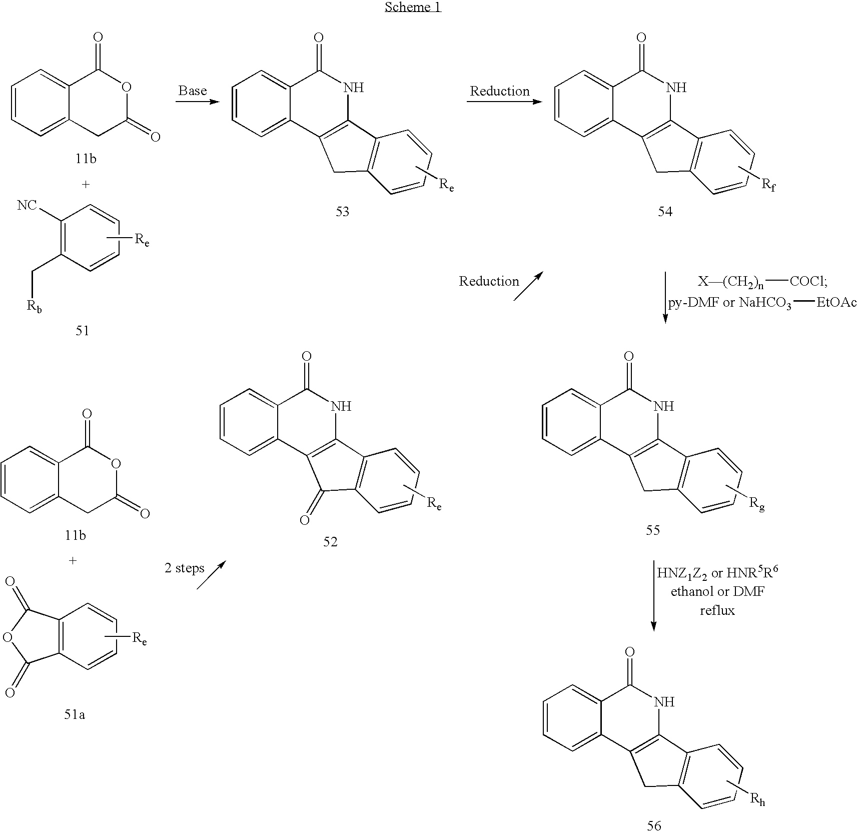

wherein: n is as defined above for Formula (I) and Formula (II);

R⁵ and R⁶ are as defined above for Formula (I);

Z₁ and Z₂ as defined above for Formula (II);

X is a leaving group such as bromide or chloride;

R_{b} is —Cl, —Br, —I, —OMs, —OTs, or —OTf;

Rₑ is —NO₂;

R_{f} is —NH₂;

R_{g} is —NHC(O)—(CH₂)ₙ—X; and

Rₕ is —NHC(O)—(CH₂)ₙ—NZ₁Z₂ or —NHC(O)—(CH₂)—N(R⁵)(R⁶).

In one embodiment, R_{b} is —Br.

General Procedure for the Preparation of Compounds of Formula 56

To a solution of homophthalic anhydride (11b) (about 1 equivalent) in a suitable solvent, such as acetonitrile, is added a compound of Formula 51 (about 1 to about 2 equivalents), follwed by a suitable base, such as triethylamine (about 1 to about 5 equivalents). The resultant reaction mixture is allowed to stir for about 1 hour, at which time a precipitate appears. The reaction mixture is then heated to reflux for about 20 hours, cooled to room temperature and filtered. The collected solid is washed with acetonitrile and dried under vacuum to provide a compound of Formula 53.

Compound 52 can be prepared from homophthalic anhydride (11b) and benzoic anhydride in two steps. Homophthalic anhydride and benzoic anhydride are reacted in a suitable solvent such as pyridine in the presence of an acid such as HCl, subsequently reacted with acetic anhydride in pyridine and heated to reflux, and then refluxed in the presence of an amine such as NH₃ in MeOH, to provide the compound of Formula 52.

To a solution of the compound of Formula 52 or 53 in a suitable solvent, such as DMF, is added a reducing agent, such as ammonium formate in the presence of palladium on carbon. The reaction mixture is heated to a temperature of about 90 to 100° C., cooled to room temperature and filtered to provide a compound of the Formula 54.

The compound of the Formula 54 can be reacted with X—(CH₂)ₙ—COCl, under conditions effective to form an amide of the Formula 55.

The compound of Formula 55 can be reacted with an amine of formula HNZ₁Z₂, or an amine of formula HNR⁵R⁶ in the presence of a solvent such as ethanol or DMF and heating to reflux, to form the compound of Formula 56.

The Isoquinoline Compounds of Formula (III) can be made using the methods described below in Scheme 2, wherein R¹-R¹⁰ are as defined above for the compounds of Formula (III).

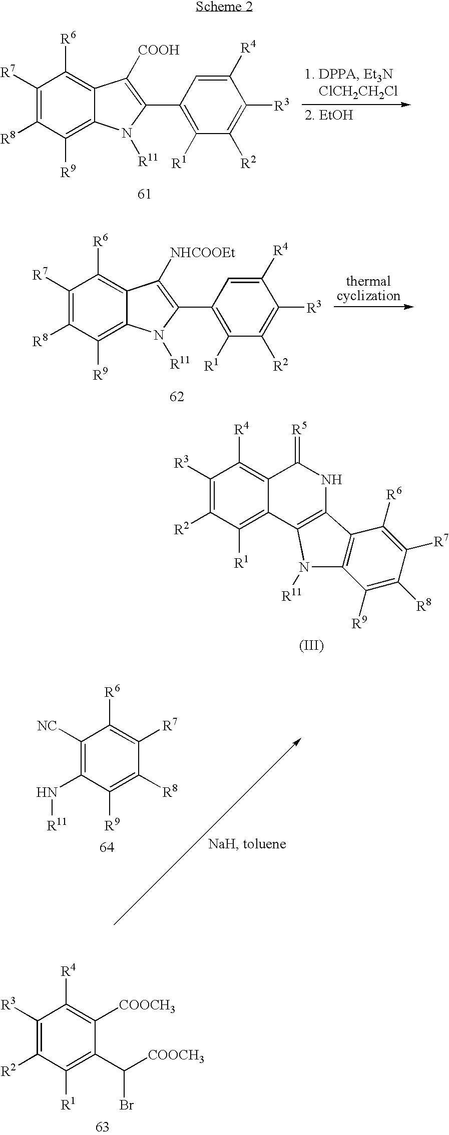

A compound of formula 61 (see Wacker et al., Tet. Lett., 43:5189-5191, 2002; and Bourdais et al, J. Het. Chem., 12:1111-1115, 1975, for methods useful to make compounds of formula 61) can be coupled with DPPA to provide the corresponding carbonate intermediates of formula 62, which can then be thermally cyclized by refluxing the compounds of formula 62 in diphenyl ether or by heating the neat compounds of formula 62 to between 300° C. and 350° C. to provide the Isoquinoline Compounds of Formula (III).

Alternatively, the Isoquinoline Compounds of Formula (III) can be made using a one pot coupling/cyclization process by reacting a bromo intermediate of formula 63 with an aromatic nitrile of formula 64 in the presence of sodium hydride.

An Isoquinoline Compound of Formula (IV) can be made by reacting a Isoquinoline Compound of Formula (III) with a compound having the formula: (a) R¹³X, where X is a leaving group such as halogen; or (b) R¹³—C(O)—O—C(O)—R¹³, under conditions well-known to those skilled in the art of organic synthesis. In either instance, R¹³ is as defined above for the compounds of Formula (IV).

### 4.8 Uses of the Isoquinoline Compounds

In accordance with the invention, the Isoquinoline Compounds are administered to a subject in need of treatment or prevention of a Condition.

### 4.5.1 Treatment or Prevention of an Inflammatory Disease

The Isoquinoline Compounds can be used to treat an inflammatory disease. Inflammatory diseases can arise where there is an inflammation of the body tissue. These include local inflammatory responses and systemic inflammation. Examples of inflammatory diseases treatable or preventable using the Isoquinoline Compounds include, but are not limited to, organ transplant rejection; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, and chronic obstructive airway disease; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimers disease, infectious meningitis, encephalomyelitis, Huntington's disease, amyotrophic lateral sclerosis and viral or autoimmune encephalitis; as well as various other diseases that can have significant inflammatory components, including preeclampsia, chronic liver failure, and brain and spinal cord trauma. The inflammatory disease can also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In one embodiment, the inflammatory disease is the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

### 4.5.2 Treatment or Prevention of a Reperfusion Injury

The Isoquinoline Compounds can be used to treat a reperfusion injury. Reperfusion refers to the process whereby blood flow in the blood vessels is resumed following ischemia, such as occurs following constriction or obstruction of the vessel. Reperfusion injury can result following a naturally occurring episode, such as a myocardial infarction, stroke, or during a surgical procedure where blood flow in vessels is intentionally or unintentionally blocked. Examples of reperfusion injuries treatable or preventable using the Isoquinoline Compounds include, but are not limited to, intestinal reperfusion injury, myocardial reperfusion injury, and reperfusion injury resulting from cardiopulmonary bypass surgery, aortic aneurysm repair surgery, carotid endarterectomy surgery, or hemorrhagic shock.

In one embodiment, the reperfusion injury results from cardiopulmonary bypass surgery, aortic aneurysm repair surgery, carotid endarterectomy surgery or hemorrhagic shock.

### 4.5.3 Treatment or Prevention of Reoxygenation Injury Resulting from Organ Transplantation

The Isoquinoline Compounds can be used to treat or prevent reoxygenation injury resulting from organ transplantation. Examples of reoxygenation injuries treatable or preventable using the Isoquinoline Compounds include, but are not limited to, transplantation of the following organs: heart, lung, liver, kidney, pancreas, intestine, and cornea.

In one embodiment, reoxygenation injury resulting from organ transplantation occurs during the organ transplantation.

### 4.5.4 Treatment or Prevention of an Ischemic Condition

The Isoquinoline Compounds can be used to treat or prevent an ischemic condition. Examples of ischemic conditions treatable or preventable using the Isoquinoline Compounds include, but are not limited to, stable angina, unstable angina, myocardial ischemia, hepatic ischemia, mesenteric artery ischemia, ischemic heart disease, intestinal ischemia, critical limb ischemia, chronic critical limb ischemia, cerebral ischemia, acute cardiac ischemia, and an ischemic disease of the central nervous system, such as stroke or cerebral ischemia.

In one embodiment, the ischemic condition is myocardial ischemia, stable angina, unstable angina, stroke, ischemic heart disease or cerebral ischemia.

### 4.5.5 Treatment or Prevention of Renal Failure

The Isoquinoline Compounds can be used to treat or prevent renal failure.

In one embodiment, the renal failure is chronic renal failure.

In another embodiment, the renal failure is acute renal failure.

### 4.5.6 Treatment or Prevention of a Vascular Disease

The Isoquinoline Compounds can be used to treat or prevent a vascular disease. Examples of vascular diseases treatable or preventable using the Isoquinoline Compounds include, but are not limited to, peripheral arterial occlusion, thromboangitis obliterans, Reynaud's disease and phenomenon, acrocyanosis, erythromelalgia, venous thrombosis, varicose veins, arteriovenous fistula, lymphedema, and lipedema.

In one embodiment, the vascular disease is a cardiovascular disease. Examples of cardiovascular diseases treatable or preventable using the Isoquinoline Compounds include, but are not limited to chronic heart failure, atherosclerosis, congestive heart failure, hypercholesterolemia, circulatory shock, cardiomyopathy, cardiac transplant, myocardial infarction, and a cardiac arrhythmia, such as atrial fibrillation, supraventricular tachycardia, atrial flutter, and paroxysmal atrial tachycardia.

In one embodiment, the cardiovascular disease is chronic heart failure.

In another embodiment, the cardiovascular disease is a cardiac arrhythmia.

In still another embodiment, the cardiac arrhythmia is atrial fibrillation, supraventricular tachycardia, atrial flutter or paroxysmal atrial tachycardia.

### 4.5.7. Treatment or Prevention of Diabetes or a Diabetic Complication

The Isoquinoline Compounds can be used to treat or prevent diabetes mellitus or its complications. Examples of diabetes treatable or preventable using the Isoquinoline Compounds include, but are not limited to, Type I diabetes (Insulin Dependent Diabetes Mellitus), Type II diabetes (Non-Insulin Dependent Diabetes Mellitus), gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), Type A insulin resistance syndrome, Type B insulin resistance syndrome, lipatrophic diabetes, and diabetes induced by β-cell toxins.

The Isoquinoline Compounds can also be used to treat or prevent a diabetic complication. Examples of diabetes mellitus or its complications that are treatable or preventable using the Isoquinoline Compounds include, but are not limited to, diabetic cataract, glaucoma, retinopathy, nephropathy, (such as microaluminuria and progressive diabetic nephropathy), polyneuropathy, gangrene of the feet, immune-complex vasculitis, systemic lupus erythematosus (SLE), atherosclerotic coronary arterial disease, peripheral arterial disease, nonketotic hyperglycemic-hyperosmolar coma, mononeuropathies, autonomic neuropathy, foot ulcers, joint problems, and a skin or mucous membrane complication (such as an infection, a shin spot, a candidal infection or necrobiosis lipoidica diabeticorumobesity), hyperlipidemia, hypertension, syndrome of insulin resistance, coronary artery disease, retinopathy, diabetic neuropathy, polyneuropathy, mononeuropathies, autonomic neuropathy, a foot ulcer, a joint problem, a fungal infection, a bacterial infection, and cardiomyopathy.

### 4.5.8 Treatment or Prevention of Parkinson's Disease

The Isoquinoline Compounds can be used to treat or prevent Parkinson's disease.

### 4.5.9 Treatment or Prevention of Cancer

The Isoquinoline Compounds can be used to treat or prevent cancer.

The invention provides methods for treating or preventing cancer, comprising administering to a subject in need of such treatment or prevention: (i) an effective amount of an Isoquinoline Compound; and (ii) an effective amount of another anticancer agent.

Examples of cancers treatable or preventable using the Isoquinoline Compounds include, but are not limited to, the cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
metastatic melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera
Central nervous system lymphomas
CNS and Brain cancers:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment the cancer is lung cancer, breast cancer, colorectal cancer, prostate cancer, a leukemia, a lymphoma, non-Hodgkin's lymphoma, skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment the cancer is metastatic cancer.

In still another embodiment, the subject in need of treatment has previously undergone or is presently undergoing treatment for cancer. The treatment includes, but is not limited to, prior chemotherapy, radiation therapy, surgery or immunotherapy, such as administration of cancer vaccines.

The Isoquinoline Compounds are also useful for the treatment or prevention of a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., El-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11): 1574-9); and Human Immune deficiency Virus (HIV) infection, which can lead to cancer as a consequence of immunodeficiency (see, e.g., Dal Maso et al., Lancet Oncol (2003) 4(2):110-9).

### 4.5.9.1 Prophylactic Methods for Cancer

The Isoquinoline Compounds can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic or therapeutic administration of an Isoquinoline Compound. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are treatable or preventable according to the present methods.

In another embodiment, fibrocystic disease (cystic hyperplasia, mammary dysplasia, particularly adenosis (benign epithelial hyperplasia)) are treatable or preventable according to the present methods.

In other embodiments, a subject that has one or more of the following predisposing factors for malignancy can be treated by administration of an effective amount of an Isoquinoline Compound: a chromosomal translocation associated with a malignancy (e.g., the Philadelphia chromosome for chronic myelogenous leukemia; t(14; 18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; and exposure to carcinogens (e.g., smoking, second-hand smoke exposure, and inhalation of or contacting with certain chemicals).

### 4.5.9.2 Combination Chemotherapy for the Treatment of Cancer

In one aspect, the present methods for treating or preventing cancer can further comprise the administration of another anticancer agent.

In one embodiment, the present invention provides methods for treating or preventing cancer, comprising the administration of an effective amount of the following to a subject in need thereof: (i) an Isoquinoline Compound and (ii) another anticancer agent.

The Isoquinoline Compound and other anticancer agent can be administered concurrently. In this embodiment the Isoquinoline Compound and other anticancer agent can be administered within the same composition, or can be administered from different compositions, via the same or different routes of administration.

In another embodiment, the Isoquinoline Compound is administered during a time when the other anticancer agent exerts its prophylactic or therapeutic effect, or vice versa.

In one embodiment, the Isoquinoline Compound or other anticancer agent are administered in doses commonly employed when such agents are used as monotherapy for the treatment of cancer.

In one embodiment, the Isoquinoline Compound or other anticancer agent are administered in doses that are lower than the doses commonly employed when such agents are used as monotherapy for the treatment of cancer.

In one embodiment, the Isoquinoline Compound and other anticancer agent act synergistically and are administered in doses that are lower than the doses commonly employed when such agents are used as monotherapy for the treatment of cancer.

The dosage of the Isoquinoline Compound or other anticancer agent administered as well as the dosing schedule can depend on various parameters, including, but not limited to, the cancer being treated, the subject's general health, and the administering physician's discretion.

An Isoquinoline Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent, to a subject in need thereof. In various embodiments an (i) Isoquinoline Compound and (ii) the other anticancer agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, an (i) Isoquinoline Compound and (ii) the other anticancer agent are administered within 3 hours. In another embodiment, an (i) Isoquinoline Compound and (ii) the other anticancer agent are administered at 1 minute to 24 hours apart.

In one embodiment, an effective amount of an Isoquinoline Compound and an effective amount of another anticancer agent are present in the same composition. In one embodiment, this composition is useful for oral administration. In another embodiment, this composition is useful for intravenous administration.

Cancers that can be treated or prevented by administering an Isoquinoline Compound and the other anticancer agent include, but are not limited to, the list of cancers set forth above in Table 1.

In one embodiment, the cancer is brain cancer.

In specific embodiments, the brain cancer is pilocytic astrocytoma, astrocytoma, anaplastic astrocytoma, glioblastoma multiforme or a metastatic brain tumor.

In one embodiment, the cancer is melanoma.

In a specific embodiment, the melanoma is metastatic melanoma.

The Isoquinoline Compound and other anticancer agent, can act additively or synergistically. A synergistic combination of an Isoquinoline Compound and the other anticancer agent, might allow the use of lower dosages of one or both of these agents and/or less frequent administration of the agents to a subject with cancer. The ability to utilize lower dosages of one or both of the Isoquinoline Compound and other anticancer agent and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, the Isoquinoline Compound and other anticancer agent act synergistically when administered in doses typically employed when such agents are used as monotherapy for the treatment of cancer. In another embodiment, the Isoquinoline Compound and other anticancer agent act synergistically when administered in doses that are lower than doses typically employed when such agents are used as monotherapy for the treatment of cancer.

In one embodiment, the administration of an effective amount of an Isoquinoline Compound and an effective amount of another anticancer agent inhibits the resistance of a cancer to the other anticancer agent. In one embodiment, the cancer is a tumor.

Suitable other anticancer agents useful in the methods and compositions of the present invention include, but are not limited to, temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agent is, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum containing complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

Other additional anticancer agents that can be used in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin-2 (including recombinant interleukin-2, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further anticancer drugs that can be used in the methods and compositions of the invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta Lactam Derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In one another embodiment, the other anticancer agent is interferon-α.

In another embodiment, the other anticancer agent is interleukin-2.

In one embodiment, the other anticancer agent is an alkylating agent, such as a nitrogen mustard, a nitrosourea, an alkylsulfonate, a triazene, or a platinum-containing agent.

In another embodiment, the other anticancer agent is a triazene alkylating agent.

In a specific embodiment, the other anticancer agent is temozolomide.

Temozolomide can be administered to a subject at dosages ranging from about 60 mg/m² (of a subject's body surface area) to about 250 mg/m² and from about 100 mg/m² to about 200 mg/m². In specific embodiments, the dosages of temozolomide are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², or about 250 mg/m².

In a particular embodiment, temozolomide is administered orally.

In one embodiment, temozolomide is administered orally to a subject at a dose ranging from about 150 mg/m² to about 200 mg/m².

In another embodiment, temozolomide is administered orally to a subject once per day for five consecutive days at a dose ranging from about 150 mg/m² to about 200 mg/m².

In a specific embodiment, temozolomide is administered orally to a subject once per day for five consecutive days at a dose ranging from about 150 mg/m² to about 200 mg/m² on days 1-5, then again orally once per day for five consecutive days on days 28-32 at a dose ranging from about 150 mg/m² to about 200 mg/m², then again orally once per day for five consecutive days on days 55-59 at a dose ranging from about 150 mg/m² to about 200 mg/m².

In a specific embodiment, the other anticancer agent is procarbazine.

Procarbazine can be administered to a subject at dosages ranging from about 50 mg/m² (of a subject's body surface area) to about 100 mg/m² and from about 60 mg/m² to about 100 mg/m². In specific embodiments, the dosages of procarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a particular embodiment, procarbazine is administered intravenously.

In one embodiment, procarbazine is administered intravenously to a subject at a dose ranging from about 50 mg/m² to about 100 mg/m².

In another embodiment, procarbazine is administered intravenously to a subject once per day for five consecutive days at a dose ranging from about 50 mg/m² to about 100 mg/m².

In a specific embodiment, procarbazine is administered intravenously to a subject once per day for five consecutive days at a dose ranging from about 50 mg/m² to about 100 mg/m² on days 1-5, then again intravenously once per day for five consecutive days on days 28-32 at a dose ranging from about 50 mg/m² to about 100 mg/m², then again intravenously once per day for five consecutive days on days 55-59 at a dose ranging from about 50 mg/m² to about 100 mg/m².

In another embodiment, procarbazine is administered intravenously once to a subject at a dose ranging from about 50 mg/m² to about 100 mg/m².

In a specific embodiment, the other anticancer agent is dacarbazine.

Dacarbazine can be administered to a subject at dosages ranging from about 60 mg/m² (of a subject's body surface area) to about 250 mg/m² and from about 150 mg/m² to about 250 mg/m². In specific embodiments, the dosages of dacarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m, about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a particular embodiment, dacarbazine is administered intravenously.

In one embodiment, dacarbazine is administered intravenously to a subject at a dose ranging from about 150 mg/m² to about 250 mg/m².

In another embodiment, dacarbazine is administered intravenously to a subject once per day for five consecutive days at a dose ranging from about 150 mg/m² to about 250 mg/m².

In a specific embodiment, dacarbazine is administered intravenously to a subject once per day for five consecutive days at a dose ranging from about 150 mg/m² to about 250 mg/m² on days 1-5, then again intravenously once per day for five consecutive days on days 28-32 at a dose ranging from about 150 mg/m² to about 250 mg/m², then again intravenously once per day for five consecutive days on days 55-59 at a dose ranging from about 150 mg/m² to about 250 mg/m².

In one embodiment, dacarbazine is administered intravenously once to a subject at a dose ranging from about 150 mg/m² to about 250 mg/m².

In one embodiment, the other anticancer agent is a Topoisomerase I inhibitor, such as etoposide, teniposide, topotecan, irinotecan, 9-aminocamptothecin, camptothecin, or crisnatol.

In a specific embodiment, the other anticancer agent is irinotecan.

Irinotecan can be administered to a subject at dosages ranging from about 50 mg/m² (of a subject's body surface area) to about 150 mg/m² and from about 75 mg/m² to about 150 mg/m². In specific embodiments, the dosages of irinotecan are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a particular embodiment, irinotecan is administered intravenously.

In one embodiment, irinotecan is administered intravenously to a subject at a dose ranging from about 50 mg/m² to about 150 mg/m².

In another embodiment, irinotecan is administered intravenously to a subject once per day for five consecutive days at a dose ranging from about 50 mg/m² to about 150 mg/m².

In a specific embodiment, irinotecan is administered intravenously to a subject once per day for five consecutive days at a dose ranging from about 50 mg/m² to about 150 mg/m² on days 1-5, then again intravenously once per day for five consecutive days on days 28-32 at a dose ranging from about 50 mg/m² to about 150 mg/m², then again intravenously once per day for five consecutive days on days 55-59 at a dose ranging from about 50 mg/m² to about 150 mg/m².

In one embodiment, the invention provides administration of an effective amount of: (i) an Isoquinoline Compound and (ii) one or more other anticancer agents.

In one embodiment, (i) an Isoquinoline Compound and (ii) one or more other anticancer agents are used as monotherapy for the treatment of cancer.

In another embodiment, (i) an Isoquinoline Compound and (ii) one or more other anticancer agents act synergistically and are administered in doses that are less than the doses commonly employed when such agents are used as monotherapy for the treatment of cancer.

The dosage of the (i) an Isoquinoline Compound and (ii) one or more other anticancer agents administered as well as the dosing schedule can depend on various parameters, including, but not limited to, the cancer being treated, the patient's general health, and the administering physician's discretion.

In one embodiment, the other anticancer agent is O-6-benzylguanine.

In another embodiment, the other anticancer agent is O-6-benzylguanine and temozolomide.

In another embodiment, the other anticancer agent is O-6-benzylguanine and procarbazine.

In still another embodiment, the other anticancer agent is O-6-benzylguanine and dacarbazine.

### 4.5.9.3 Multi-Therapy for Cancer

The Isoquinoline Compounds can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof (a) an amount of an Isoquinoline Compound effective to treat or prevent cancer; and (b) another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering (i) an effective amount of an Isoquinoline Compound and (ii) radiation therapy. The radiation therapy can be administered concurrently with, prior to, or subsequent to the Isoquinoline Compound, in one embodiment at least an hour, five hours, 12 hours, a day, a week, a month, in another embodiment several months (e.g., up to three months), prior or subsequent to administration of the Isoquinoline Compounds.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, the invention provides methods of treatment of cancer using an Isoquinoline Compound as an alternative to chemotherapy or radiation therapy where the chemotherapy or the radiation therapy results in negative side effects in the subject being treated. The subject being treated can, optionally, be treated with another anticancer therapy such as surgery, radiation therapy, or immunotherapy.

The Isoquinoline Compounds can also be used in vitro or ex vivo, such as for the treatment of certain cancers, including, but not limited to leukemias and lymphomas, such treatment involving autologous stem cell transplants. This can involve a process in which the subject's autologous hematopoietic stem cells are harvested and purged of all cancer cells, the subject's remaining bone-marrow cell population is then eradicated via the administration of an Isoquinoline Compound and/or radiation, and the resultant stem cells are infused back into the subject. Supportive care can be subsequently provided while bone marrow function is restored and the subject recovers.

### 4.6 Therapeutic/Prophylactic Administration and Compositions of the Invention

Due to their activity, the Isoquinoline Compounds are advantageously useful in veterinary and human medicine. As described above, the Isoquinoline Compounds are useful for treating or preventing a Condition in a subject in need thereof.

The Isoquinoline Compounds can be administered in amounts that are effective to treat or prevent a Condition in a subject.

When administered to a subject, the Isoquinoline Compounds can be administered as a component of a composition that comprises a physiologically acceptable carrier or vehicle. The present compositions, which comprise an Isoquinoline Compound, can be administered orally. The Isoquinoline Compounds can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of an Isoquinoline Compound into the bloodstream.

In one embodiment, the Isoquinoline Compounds are administered orally.

In other embodiments, it can be desirable to administer the Isoquinoline Compounds locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Isoquinoline Compounds into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the Isoquinoline Compounds can be formulated as a suppository, with traditional binders and excipients such as triglycerides.

In another embodiment the Isoquinoline Compounds can be delivered in a vesicle, in particular a liposome (see Langer, Science 249:1527-1533 (1990) and Treat or prevent et al., Liposomes in Therapy of Infectious Disease and Cancer 317-327 and 353-365 (1989)).

In yet another embodiment the Isoquinoline Compounds can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Isoquinoline Compounds, e.g., the spinal column, brain, skin, lung, or gastrointestinal tract, thus requiring only a fraction of the systemic dose.

The present compositions can optionally comprise a suitable amount of a pharmaceutically acceptable excipient so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to a subject. Water is a particularly useful excipient when the Isoquinoline Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment the Isoquinoline Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving an Isoquinoline Compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Isoquinoline Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Isoquinoline Compounds are to be administered by infusion, they can be dispensed, for example, with an infusion bottle containing sterile pharmaceutical grade water or saline. Where the Isoquinoline Compounds are administered by injection, an ampule of sterile water for injection or saline can be provided so that the ingredients can be mixed prior to administration.

The Isoquinoline Compounds can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354;556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of an Isoquinoline Compound to treat or prevent the Condition in a minimal amount of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the Isoquinoline Compound, and can thus reduce the occurrence of adverse side effects.

Controlled- or sustained-release compositions can initially release an amount of an Isoquinoline Compound that promptly produces the desired therapeutic or prophylactic effect, and gradually and continually release other amounts of the Isoquinoline Compound to maintain this level of therapeutic or prophylactic effect over an extended period of time. To maintain a constant level of the Isoquinoline Compound in the body, the Isoquinoline Compound can be released from the dosage form at a rate that will replace the amount of Isoquinoline Compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the Isoquinoline Compound that is effective in the treatment or prevention of a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Isoquinoline Compound is administered, the effective dosage amounts correspond to the total amount administered.

Compositions can be prepared according to conventional mixing, granulating or coating methods, respectively, and the present compositions can contain, in one embodiment, from about 0.1% to about 99%; and in another embodiment from about 1% to about 70% of the Isoquinoline Compound by weight or volume.

The dosage regimen utilizing the Isoquinoline Compound can be selected in accordance with a variety of factors including type, species, age, weight, sex and medical condition of the subject; the severity of the condition to be treated; the route of administration; the renal or hepatic function of the subject; and the particular Isoquinoline Compound employed. A person skilled in the art can readily determine the effective amount of the drug useful for treating or preventing the Condition.

The Isoquinoline Compounds can be administered in a single daily dose, or the total daily dosage can be administered in divided doses of two, three or four times daily. Furthermore, the Isoquinoline Compounds can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Isoquinoline Compound ranges from about 0.1% to about 15%, w/w or w/v.

In one embodiment, the compositions comprise an amount of (i) an Isoquinoline Compound and (ii) the other anticancer agent which together are effective to treat or prevent cancer. In another embodiment, the amount of (i) an Isoquinoline Compound and (ii) the other anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of combined amount of (i) an Isoquinoline Compound and (ii) the other anticancer agent by weight of the composition. Other compositions of the present invention are prepared so that a parenteral dosage unit contains from about 0.01% to about 2% by weight of the composition.

The Isoquinoline Compounds can be assayed in vitro or in vivo for the desired therapeutic or prophylactic activity prior to use in humans. Animal model systems can be used to demonstrate safety and efficacy.

The present methods for treating or preventing a Condition in a subject in need thereof can further comprise administering another prophylactic or therapeutic agent to the subject being administered an Isoquinoline Compound. In one embodiment the other prophylactic or therapeutic agent is administered in an effective amount. The other prophylactic or therapeutic agent includes, but is not limited to, an anti-inflammatory agent, an anti-renal failure agent, an anti-diabetic agent, and anti-cardiovascular disease agent, an antiemetic agent, a hematopoietic colony stimulating factor, an anxiolytic agent, and an analgesic agent.

In one embodiment, the other prophylactic or therapeutic agent is an agent useful for reducing any potential side effect of an Isoquinoline Compound. Such potential side effects include, but are not limited to, nausea, vomiting, headache, low white blood cell count, low red blood cell count, low platelet count, headache, fever, lethargy, muscle aches, general pain, bone pain, pain at an injection site, diarrhea, neuropathy, pruritis, mouth sores, alopecia, anxiety or depression.

In one embodiment, the Isoquinoline Compound can be administered prior to, concurrently with, or after an anti-inflammatory agent, or on the same day, or within 1 hour, 2 hours, 12 hours, 24 hours, 48 hours or 72 hours of each other.

In another embodiment, the Isoquinoline Compound can be administered prior to, concurrently with, or after an anti-renal failure agent, or on the same day, or within 1 hour, 2 hours, 12 hours, 24 hours, 48 hours or 72 hours of each other.

In still another embodiment, the Isoquinoline Compound can be administered prior to, concurrently with, or after an anti-diabetic agent, or on the same day, or within 1 hour, 2 hours, 12 hours, 24 hours, 48 hours or 72 hours of each other.

In yet another embodiment, the Isoquinoline Compound can be administered prior to, concurrently with, or after an anti-cardiovascular disease agent, or on the same day, or within 1 hour, 2 hours, 12 hours, 24 hours, 48 hours or 72 hours of each other.

In a further embodiment, the Isoquinoline Compound can be administered prior to, concurrently with, or after an antiemetic agent, or on the same day, or within 1 hour, 2 hours, 12 hours, 24 hours, 48 hours or 72 hours of each other.

In another embodiment, the Isoquinoline Compound can be administered prior to, concurrently with, or after a hematopoietic colony-stimulating factor, or on the same day, or within 1 hour, 2 hours, 12 hours, 24 hours, 48 hours, 72 hours, 1 week, 2 weeks, 3 weeks or 4 weeks of each other.

In still embodiment, the Isoquinoline Compound can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the Isoquinoline Compound can be administered prior to, concurrently with, or after an anxiolytic agent, or on the same day, or within 1 hour, 2 hours, 12 hours, 24 hours, 48 hours or 72 hours of each other.

Effective amounts of the other prophylactic or therapeutic agents are well known to those skilled in the art. However, it is well within the skilled artisan's purview to determine the other prophylactic or therapeutic agent's optimal effective amount range. In one embodiment of the invention, where another prophylactic or therapeutic agent is administered to a subject, the effective amount of the Isoquinoline Compound is less than its effective amount would be where the other prophylactic or therapeutic agent is not administered. In this case, without being bound by theory, it is believed that the Isoquinoline Compounds and the other prophylactic or therapeutic agent act synergistically to treat or prevent a Condition.

Anti-inflammatory agents useful in the methods of the present invention include but are not limited to adrenocorticosteroids, such as cortisol, cortisone, fludrocortisone, prednisone, prednisolone, 6α-methylprednisolone, triamcinolone, betamethasone, and dexamethasone; and non-steroidal anti-inflammatory agents (NSAIDs), such as aspirin, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, rofecoxib, celecoxib, etodolac, and nimesulide.

Anti-renal failure agents useful in the methods of the present invention include include but are not limited to ACE (angiotensin-converting enzyme) inhibitors, such as captopril, enalaprilat, lisinopril, benazepril, fosinopril, trandolapril, quinapril, and ramipril; diuretics, such as mannitol, glycerin, furosemide, toresemide, tripamide, chlorothiazide, methyclothiazide, indapamide, amiloride, and spironolactone; and fibric acid agents, such as clofibrate, gemfibrozil, fenofibrate, ciprofibrate, and bezafibrate.

Anti-diabetic agents useful in the methods of the present invention include include but are not limited to glucagons; somatostatin; diazoxide; sulfonylureas, such as tolbutamide, acetohexamide, tolazamide, chloropropamide, glybenclamide, glipizide, gliclazide, and glimepiride; insulin secretagogues, such as repaglinide, and nateglinide; biguanides, such as metformin and phenformin; thiazolidinediones, such as pioglitazone, rosiglitazone, and troglitazone; and α-glucosidase inhibitors, such as acarbose and miglitol.

Anti-cardiovascular disease agents useful in the methods of the present invention include include but are not limited to carnitine; thiamine; and muscarinic receptor antagonists, such as atropine, scopolamine, homatropine, tropicamide, pirenzipine, ipratropium, tiotropium, and tolterodine.

Antiemetic agents useful in the methods of the present invention include include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and mixtures thereof.

Hematopoietic colony stimulating factors useful in the methods of the present invention include, but are not limited to, filgrastim, sargramostim, molgramostim and epoietin alfa.

Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Non-opioid analgesic agents useful in the methods of the present invention include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

### 4.7 Kits

The invention encompasses kits that can simplify the administration of an Isoquinoline Compound to a subject.

A typical kit of the invention comprises a unit dosage form of an Isoquinoline Compound. In one embodiment the unit dosage form is a container, which can be sterile, containing an effective amount of an Isoquinoline Compound and a physiologically acceptable carrier or vehicle. The kit can further comprise a label or printed instructions instructing the use of the Isoquinoline Compound to treat or prevent a Condition. The kit can also further comprise a unit dosage form of another prophylactic or therapeutic agent, for example, a container containing an effective amount of the other prophylactic or therapeutic agent. In one embodiment the kit comprises a container containing an effective amount of an Isoquinoline Compound and an effective amount of another prophylactic or therapeutic agent. Examples of other prophylactic or therapeutic agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag.

The invention is further described in the following examples, which do not limit the scope of the invention described in the claims. The following examples can demonstrate the usefulness of the Isoquinoline Compounds for treating or preventing a Condition.

### 5. EXAMPLES

### a) General Methods

Proton and ¹³C nuclear magnetic resonance (¹H and ¹³C-NMR) spectra were obtained from Varian 300 MHz spectrophotometer and chemical shift data are reported in parts per million, δ. Thin layer chromatography, TLC, was carried out on precoated TLC plates with silica gel 60 F-254 and preparative TLC on precoated Whatman 60A TLC plates. All intermediates and final compounds were characterized on the basis of NMR (¹H or ¹³C) and Mass spectral (MS) or elemental analysis data. Elemental analyses performed at Robertson Microlit. Lab. (Madison, N.J.) and provided results for the elements stated with +0.4% of the theoretical values. Analytical HPLC was performed using a Waters Alliance 2795 series system, equipped with a Waters UV 2996PAD (set at 254 nM) and Micromass MS Quattro LC detector or using Waters Alliance 2690 series system, equipped with the Micromass LCT detector. A YMC-Pack-ODS-AQ (series AQ12505-1546WT, size 150 mm×4.6 mm, S-5 μM) column was used. Typically, a gradient mobile phase starting with 70% water with 0.05% ammonium formate and 30% methanol with 0.05% ammonium formate (or 70% water with 0.1% TFA and 30% MeCN or 70% water with 0.1% TFA and MeOH) for 2 min, then 10% water with 0.05% ammonium formate and 90% methanol with 0.05% ammonium formate up to 10 min., then 70% water with 0.05% ammonium formate and 30% methanol with 0.05% ammonium formate thereafter was used. Flow rate 0.8 ml/min.

### b) Preparation of 5,6-Dihydro-5-oxo-9-nitro-indeno[1,2-c]isoquinoline (53a)

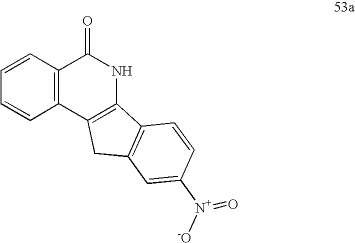

To a refluxing mixture of 2-methyl-4-nitro-benzonitrile (32.4 g, 0.2 mol) and NBS (44.470 g, 0.25 mol) in CCl₄ (300 ml) was added AIBN (0.325 g) and the resultant reaction mixture was refluxed for 4 hours. The reaction mixture was treated with AIBN (0.325 g, 31 mmol) and refluxed further for 4 hours. The reaction mixture was filtered, and the filtered succinimide was washed with CCl₄. The filtrate was concentrated in vacuo to provide a bromo compound (46 g). The bromo compound was dissolved in MeCN (200 ml), and to the reaction mixture was added homophthalic anhydride (30.780 g, 0.19 mol) at room temperature and under inert atmosphere. The reaction mixture was then treated with a solution of triethylamine (84 ml, 0.6 mol) in acetonitrile (100 ml). The reaction mixture was refluxed for 8 hours. The precipitate that formed was removed by filtration and washed with MeCN (100 ml). The washed precipitate was suspended in DMF (300 ml), which was heated at 130° C., then cooled and filtered. The resultant solid was washed with DMF (100 ml) and dried under vacuum to provide Compound 53a as a pale yellow solid (18.310 g, 33%). ¹H-NMR (DMSO-d₆): δ, 4.09 (s, 2H), 7.56 (m, 1H), 7.81-7.82 (m, 2H), 8.17 (d, J=8.4 Hz, 1H), 8.26-8.34 (m, 2H), 8.44 (s, 1H), 12.47 (s, 1H).

### c) Preparation of 5,6-Dihydro-5-oxo-9-amino-indeno[1,2-c]isoquinoline (54a)

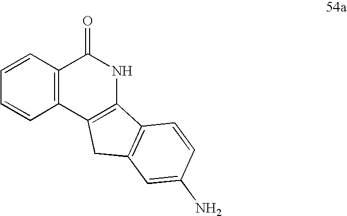

To a suspension of Compound 53a (5.3 g, 0.019 mol) and ammonium formate (5.985 g, 0.095 mol) in DMF (100 ml) was added Pd—C (5%, 100 mg) at 80° C. The reaction mixture was stirred at 100° C. for 1 hour. After the reaction mixture became clear, it was filtered through the pad of celite. The celite was washed with DMF. The filtrate was then diluted with ice, and the resultant solid was filtered, washed with water and dried at 80° C. under vacuum to provide Compound 54a (3.2 g, 68%). ¹H-NMR (DMSO-d₆): δ, 3.89 (s, 2H), 7.18 (d, J=8.4 Hz, 1H), 7.40-7.45 (m, 2H), 7.66-7.72 (m, 2H), 7.94 (d, J=8.1 Hz, 1H), 8.21 (d, J=8.1 Hz, 1H), 12.28 (s, 1H).

### d) Preparation of N-[5,6-Dihydro-5-oxo-indeno[1,2-c]isoquinolin-9-yl]-4-bromo-butylamide (55a)

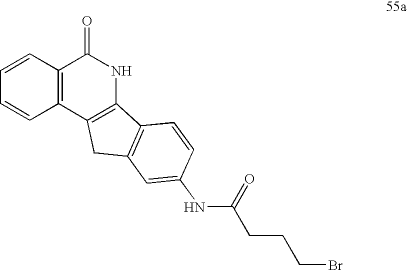

To a suspension of Compound 54a (1.5 g, 0.006 mol) in saturated NaHCO₃ (150 ml) and ethyl acetate (100 ml) was added 4-bromobutyryl chloride (5 eq). The reaction mixture was stirred at room temperature for 1 hour. The resultant solid was isolated by filtration, washed with water and ethyl acetate, and dried under vacuum to provide Compound 55a (1.625 g, 68%). ¹H-NMR (DMSO-d₆): δ, 2.09-2.13 (m, 2H), 2.47-2.52 (m, 2H), 3.58 (t, J=6.6 Hz, 2H), 3.85 (s, 2H), 7.40 (t, J=6.3 Hz, 1H), 7.50 (d, J=8.4 Hz, 1H), 7.66-7.71 (m, 2H), 7.86 (d, J=8.4 Hz, 1H), 7.92 (s, 1H), 8.20 (d, J=8.1 Hz, 1H), 10.10 (s, 1H), 12.24 (s, 1H).

### e) Preparation of N-[5,6-Dihydro-5-oxo-indeno[1,2-c]isoquinolin-9-yl]-4-chloro-butylamide (55b)

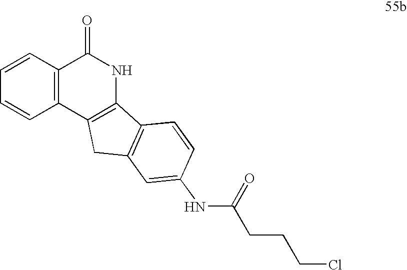

Compound 55b (N-[5,6-dihydro-5-oxo-indeno[1,2-c]isoquinolin-9-yl]-4-chloro-butylamide) was made according to the method for making Compound 55a, except that chlorobutyryl chloride was used in place of 4-bromobutyryl chloride. ¹H-NMR (DMSO-d₆): δ, 1.99-2.08 (m, 2H), 2.47-2.52 (m, 2H), 3.70 (t, J=6.6 Hz, 2H), 3.86 (s, 2H), 7.38-7.44 (m, 1H), 7.50 (d, J=8.1 Hz, 1H), 7.66-7.71 (m, 2H), 7.86 (d, J=8.1 Hz, 1H), 7.95 (s, 1H), 8.21 (d, J=8.1 Hz, 1H), 10.09 (s, 1H), 12.24 (s, 1H).

### f) Preparation of N-[5,6-Dihydro-5-oxo-indeno[1,2-c]isoquinolin-9-yl]-2-chloro-acetamide (55c)

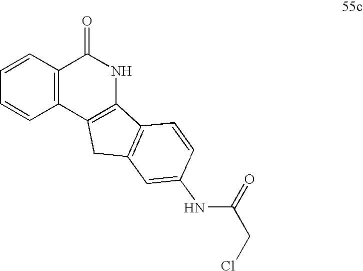

To a suspension of Compound 54a (11.5 g, 0.0060 mol) in saturated NaHCO₃ (250 ml) and ethyl acetate (250 ml) was added chloroacetyl chloride (5 eq). The reaction mixture was stirred at room temperature for 1 hour. The resultant solid was isolated by filtration; washed sequentially with ethyl acetate, water and methanol; and dried under vacuum to provide Compound 55c (1.6 g, 82%). ¹H-NMR (DMSO-d₆): δ, 3.89 (s, 2H), 4.27 (s, 2H), 7.40-7.45 (dd, J=6.3 and 8.1 Hz, 1H), 7.52 (d, J=8.1 Hz, 1H), 7.67-7.75 (m, 2H), 7.90 (d, J=8.4 Hz, 1H), 7.94 (s, 1H), 8.21 (d, J=8.1 Hz, 1H), 10.43 9s, 1H), 12.28 (s, 1H).

### g) Preparation of 2-[4-(4-Fluoro-phenyl)-piperazin-1-yl]-N-(5-oxo-5,11-dihydro-6H-indeno[1,2-c]isoquinolin-9-yl)-acetamide (11a)

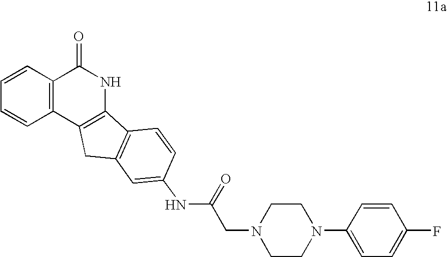

A suspension of Compound 55c (100 mg) and 4-(p-F-phenyl)piperazine in methanol (10 ml) was refluxed for overnight. The reaction mixture was allowed to cool to room temperature. The resultant solid was filtered, washed with methanol, and dried under vacuum to provide Compound 11a (120 mg). ¹H-NMR (DMSO-d₆): δ, 2.67-2.71 (m, 4H), 3.16-3.19 (m, 2H), 3.27-3.30 (m, 2H), 3.87 (s, 2H), 6.92-7.06 (m, 4H), 7.40-7.44 (dd, J=6 and 6.6 Hz, 1H), 7.60 (d, J=8.4 Hz, 1H), 7.70-7.73 (m, 2H), 7.89 (d, J=8.1 Hz, 1H), 8.0 (s, 1H), 8.22 (d, J=8.1 Hz, 1H), 9.87 (s, 1H), 12.23 (s, 1H); MS (ES⁺): m/z 469.3 (M+1).

### h) Preparation of N-(5-Oxo-5,11-dihydro-6H-indeno[1,2-c]isoquinolin-9-yl)-2-(4-phenyl-3,6-dihydro-2H-pyridin-1-yl)-acetamide (12a)

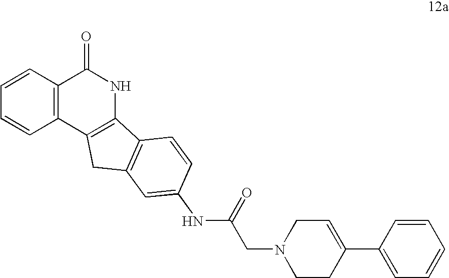

A suspension of Compound 55c (85 mg), triethylamine (1.2 eq), and 4-(phenyl)-1,2,5,6-tetrahydropyridine (62 mg) in DMF (15 ml) was heated at 60° C. for 16 hr. The reaction mixture was allowed to cool to room temperature. The resultant solid was filtered, washed with methanol, and dried under vacuum to provide Compound 12a (85 mg).

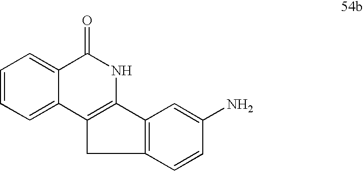

### i) 5,6-Dihydro-5-oxo-8-amino-indeno[1,2-c]isoquinoline (54b)

To a suspension of 5,6-dihydro-5-oxo-8-nitro-indeno[1,2-c]isoquinoline (1 g, 0.003 mol) and ammonium formate (5 eq) in DMF (25 ml) was added Pd—C (5%, 100 mg) at 120° C. Then the reaction mixture was stirred at 100° C. for 2 h. After the reaction mixture became clear, it was filtered through the pad of celite. The celite was washed with DMF. The filtrate was then diluted with the crushed ice and the resultant solid was filtered. The filtered solid was washed with water and dried at 80° C. under vacuum to Compound 54b (710 mg, 95%).

¹H-NMR (DMSO-d₆): δ, 3.67 (s, 2H), 5.05 (s, 2H), 6.54 (d, J=8.1 Hz, 1H), 7.16 (d, J=1.5 Hz, 1H), 7.20 (d, J=8.4 Hz, 1H), 7.36-7.41 (dd, J=6.6 and 1.5 Hz, 1H), 7.62-7.71 (m, 2H), 8.18 (d, J=7.8 Hz, 1H), 12.51 (s, 1H); MS (ES⁺): m/z 249.1 (M+1).

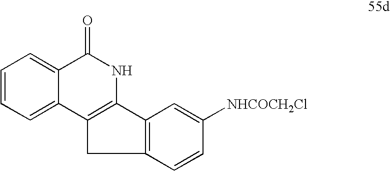

### j) N-[5,6-Dihydro-5-oxo-indeno[1,2-c]isoquinolin-8-yl]-2-chloro-acetamide (55d)

To a suspension of 5,6-dihydro-5-oxo-8-amino-indeno[1,2-c]isoquinoline 54b (500 mg, 0.002 mol) in saturated NaHCO₃ (100 ml) and ethyl acetate (25 ml) was added chloroacetyl chloride (10 eq). The reaction mixture was then stirred at room temperature for 2 h. The resultant solid was isolated by filtration and washed with ethyl acetate, water and finally with methanol. The solid was dried under vacuum to provide Compound 55d (440 mg, 67%). ¹H-NMR (DMSO-d₆): δ, 3.84 (s, 2H), 4.28 (s, 2H), 7.42-7.54 (m, 3H), 7.71-7.74 (m, 2H), 7.92 9s, 1H), 8.20-8.23 (m, 2H), 10.36 (s, 1H), 12.41 (s, 1H).

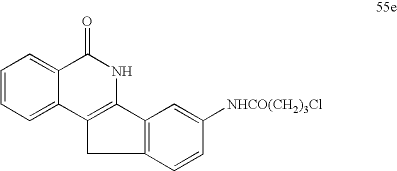

### k) N-[5,6-Dihydro-5-oxo-indeno[1,2-c]isoquinolin-8-yl]-4-chloro-butylamide (55e)

Similarly, N-[5,6-dihydro-5-oxo-indeno[1,2-c]isoquinolin-8-yl]-4-chloro-butylamide (Compound 55e) was prepared from Compound 54b using chlorobutyryl chloride in the presence of aqueous NaHCO₃ and ethyl acetate in 97% yield. ¹H-NMR (DMSO-d₆): δ, 2.01-2.05 (t, J=6.6 Hz, 2H), 2.46-2.51 (m, 2H), 3.70 (t, J=6.3 Hz, 2H), 3.81 (s, 2H), 7.42-7.49 (m, 3H), 7.68-7.72 (m, 2H), 8.18-8.22 (m, 2H), 10.01 (s, 1H), 12.36 (s, 1H).

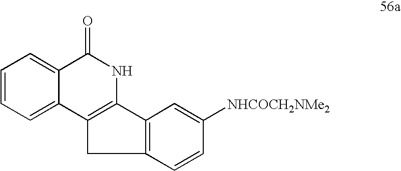

### l) N-[5,6-Dihydro-5-oxo-indeno[1,2-c]isoquinolin-8-yl]-N,N-dimethylacetamide (56a)

A suspension of N-[5,6-dihydro-5-oxo-indeno[1,2-c]isoquinolin-9-yl]-chloroacetamide (Compound 55d) (100 mg, 0.0003 mol) in dimethyl amine in ethanol (2N, 10 ml) was refluxed for 24 h. The reaction mixture was cooled to room temperature and treated with acid, then base. The resultant solid was filtered and washed with ethanol and dried under vacuum to provide Compound 56a (72 mg, 75%). ¹H-NMR (DMSO-d₆): δ, 2.27 (s, 6H), 3.07 (s, 2H), 3.81 (s, 2H), 7.40-7.48 (m, 3H), 7.67-7.70 (m, 2H), 8.17-8.22 (m, 2H), 9.27 (s, 1H), 12.3 (s, 1H). MS (ES⁺): m/z 334.0 (M+1)

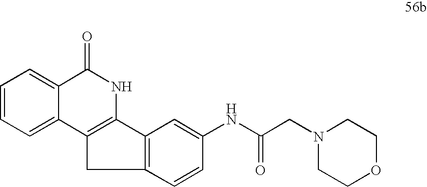

### m) N-[5,6-Dihydro-5-oxo-indeno[1,2-c]isoquinolin-8-yl]-4-morpholino-acetamide (56b)

A suspension of N-[5,6-dihydro-5-oxo-indeno[1,2-c]isoquinolin-9-yl]-chloroacetamide (Compound 55d) (200 mg, 0.0006 mol) in morpholine (5 ml) and methanol (10 ml) was refluxed for 8 h. The reaction mixture was cooled to room temperature and the resultant solid was filtered and washed with methanol. The solid was dried under vacuum to provide Compound 56b (152 mg, 66%).

¹H-NMR (DMSO-d₆): δ, 2.50-2.52 (m, 4H), 3.13 (s, 2H), 3.61-3.63 (m, 4H), 3.82 (s, 2H), 7.40-7.51 (m, 3H), 7.70-7.72 (m, 2H), 8.16 (s, 1H), 8.21 (d, J=8.1 Hz, 1H), 9.73 (s, 1H), 12.33 (s, 1H); Anal. Calcd for C₂₂H₂₁N₃O₃: C, 70.38; H, 5.64; N, 11.19. Found: C, 69.99; H, 5.76; N, 11.13.

### n) N-[5,6-Dihydro-5-oxo-indeno[1,2-c]isoquinolin-8-yl]-4-N-methyl-piperazino-acetamide (56c)

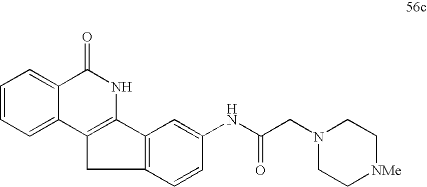

Similarly, N-[5,6-dihydro-5-oxo-indeno[1,2-c]isoquinolin-8-yl]-4-methylpiperazine-acetamide (Compound 56c) was prepared from Compound 55d using 4-methylpiperazine and methanol in 67% yield. ¹H-NMR (DMSO-d₆): δ, 2.16 (s, 3H), 2.30-2.41 (m, 4H), 2.50-5.53 (m, 4H), 3.11 (s, 2H), 3.82 (s, 2H), 7.40-7.45 (m, 1H), 7.50 (d, J=8.1 Hz, 1H), 7.56 (d, J=8.1 Hz, 1H), 7.68-7.75 (m, 2H), 8.11 (s, 1H), 8.20 (d, J=8.1 Hz, 1H), 9.64 (s, 1H), 12.32 (s, 1H); Anal. Calcd for C₂₃H₂₄N₄O₂: C, 71.11; H, 6.23; N, 14.42. Found: C, 70.87; H, 6.22; N, 14.39.

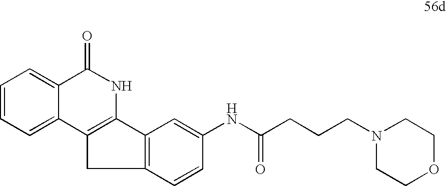

### o) N-[5,6-Dihydro-5-oxo-indeno[1,2-c]isoquinolin-8-yl]-4-morpholino-butylamide (56d)

A suspension of N-[5,6-dihydro-5-oxo-indeno[1,2-c]isoquinolin-8-yl]-4-chloro-butylamide (75 mg, 0.0002 mol) in morpholine (5 ml) was refluxed overnight. The reaction mixture was cooled to room temperature and the resultant solid was filtered. The solid was washed with methanol and dried under vacuum to provide Compound 56d (70 mg, 82%). ¹H-NMR (DMSO-d₆): δ, 1.72-1.76 (t, J=6 Hz, 2H), 2.27-2.37 (m, 8H), 3.5-3.53 (m, 4H), 3.81 (s, 2H), 7.40-7.49 (m, 3H), 7.71-7.2 (m, 2H), 8.19-8.21 (m, 2H), 9.90 (s, 1H), 12.36 (s, 1H); MS (ES⁺): m/z 404.28 [M+1]; Anal. Calcd for C₂₄H₂₅N₃O₃: C, 71.44; H, 6.25; N, 10.41. Found: C, 71.05; H, 6.19; N, 10.17.

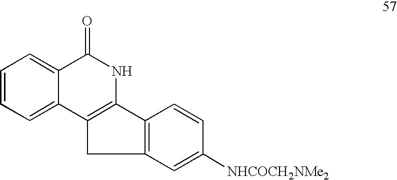

### p) N-[5,6-Dihydro-5-oxo-indeno[1,2-c]isoquinolin-9-yl]-N,N-dimethylacetamide (57)

A suspension of N-[5,6-dihydro-5-oxo-indeno[1,2-c]isoquinolin-9-yl]-chloroacetamide (Compound 55c) (1.6 g, 0.0049 mol) in dimethyl amine in ethanol (2N, 200 ml) was refluxed for 24 h. Additional solution of dimethyl amine in ethanol (2N, 200 ml) was added and the reaction mixture was refluxed further for 24 h. The reaction mixture was cooled to room temperature and the resultant solid was filtered and washed with ethanol. The solid was dried under vacuum to provide Compound 57 (1.510 g, 92%).

¹H-NMR (DMSO-d₆): δ, 2.27 (s, 6H), 3.07 (s, 2H), 3.85 (s, 2H), 7.38-7.43 (m, 1H), 7.58 (d, J=8.1 Hz, 1H), 7.66-7.73 (m, 2H), 7.87 (d, J=8.1 Hz, 1H), 8.02 (s, 1H), 8.20 (d, J=8.1 Hz, 1H), 9.82 (s, 1H), 12.21 (s, 1H); MS (ES⁺): m/z 334.01 (M+1).

### q) N-[5,6-Dihydro-5-oxo-indeno[1,2-c]isoquinolin-9-yl]-N,N-dimethylacetamidecamphor sulfonic acid salt (57a)

To a suspension of N-[5,6-dihydro-5-oxo-indeno[1,2-c]isoquinolin-9-yl]-N,N-dimethylacetamide (Compound 57) (1.250 g, 0.0037 mol) in MeOH (200 ml) was added camphorsulfonic acid (0.915 g, 0.0039 mol). The reaction mixture was stirred at room temperature for 1 hour and was concentrated on rotavaporator. The resultant residue was dissolved in DI water (300 ml). The resultant solution was filtered and treated with decolorising charcoal (1 g) and stirred at 100 to 105° C. for 30 min. The resultant solution was filtered through the pad of celite and celite was washed with water. The filtrate was then concentrated on freeze dryer to provide N-[5,6-dihydro-5-oxo-indeno[1,2-c]isoquinolin-9-yl]-N,N-dimethylaminoacetamide camphor sulfonic acid salt (Compound 57a) (1.660 g, 75%).

¹H-NMR (DMSO-d₆): δ, 0.72 (s, 3H), 1.02 9s, 3H), 1.20-1.30 (m, 2H), 1.74-1.92 (m, 3H), 2.17-2.25 (m, 1H), 2.35 (d, J=14.7 Hz, 1H), 2.64 (t, J=9.9 Hz, 1H, 2.80 (d, J=14.7 Hz, 1H), 3.90 (s, 2H), 4.16 (s, 2H), 7.41-7.46 (dd, J=6.3 and 8.1 hz, 1H), 7.53 (d, J=8.1 Hz, 1H), 7.68-7.73 (m, 2H), 7.92-7.94 (m, 2H), 8.22 (d, J=8.1 Hz, 1H), 9.77 (s, 1H), 10.68 (s, 1H), 12.29 (s, 1H). MS (ES⁺): m/z 334.22 (M+1).

### r) N-[5,6-Dihydro-5-oxo-indeno[1,2-c]isoquinolin-9-yl]-4-morpholino-butylamide (58)

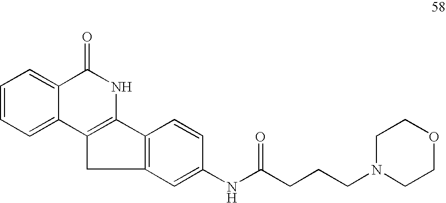

To a suspension of N-[5,6-dihydro-5-oxo-indeno[1,2-c]isoquinolin-9-yl]-4-bromobutylamide (Compound 55a) (1.625 g, 0.004 mol) in DMF (25 ml) was added triethylamine (5 ml) followed by morpholine (5 ml). The reaction mixture was heated at 140 to 155° C. for 1 h, cooled to room temperature and stirred overnight. The resultant solid was filtered and washed with DMF, water and finally with methanol. The resultant solid was dried under vacuum to provide Compound 58 (1.380 g, 85%).

¹H-NMR (DMSO-d₆): δ, 1.72-1.76 (dd, J=6.9 and 7.2 Hz, 2H), 2.26-2.37 (m, 8H), 3.51-3.54 (t, J=4.2 Hz, 4H), 3.86 (s, 2H), 7.39-7.43 (dd, J=6.3 and 6.6 Hz, 1H), 7.51 (d, J=6.6 Hz, 1H), 7.66-7.74 (m, 2H), 7.86 (d, J=8.4 Hz, 1H), 7.96 (s, 1H), 8.20 (d, J=8.1 Hz, 1H), 10.0 (s, 1H), 12.25 (s, 1H).

### s) N-[5,6-Dihydro-5-oxo-indeno[1,2-c]isoquinolin-9-yl]-4-morpholino-butylamide camphor sulfonic acid (58a)

To a suspension of N-[5,6-dihydro-5-oxo-indeno[1,2-c]isoquinolin-9-yl]-4-morpholino-butylamide (Compound 58) (0.403 g, 0.001 mol) in MeOH (20 ml) was added camphor sulfonic acid (255 mg, 0.0011 mol). The reaction mixture was stirred at room temperature for 2 hr, and concentrated on rotavaporator. The resultant residue was dissolved in DI water (40 ml), treated with decolorising charcoal (0.5 g) and stirred at 90 to 100° C. for 30 min. The resultant solution was filtered through the pad of celite and celite was washed with water. The filtrate was then concentrated on freeze dryer to provide N-[5,6-dihydro-5-oxo-indeno[1,2-c]isoquinolin-9-yl]-4-morpholino-butylamide camphor sulfonic acid salt (Compound 58a) (0.450 g, 71%).

¹H-NMR (DMSO-d₆): δ, 0.72 (s, 3H), 1.02 (s, 3H), 1.20-1.30 (m, 2H), 1.76 (d, J=18 Hz, 1H), 1.82-1.86 (m, 1H), 1.89-1.97 (m, 3H), 1.99-2.25 (m, 1H), 2.35 (d, J=14.7 Hz, 1H), 2.43-2.48 (m, 2H), 2.64-2.71 (dd, J=11.7 and 14.7 Hz, 1H), 2.85 (d, J=14.7 Hz, 1H), 3.05-3.13 (m, 4H), 3.46 (d, J=11.7 Hz, 2H), 3.64 (t, J=12 Hz, 2H), 3.86 (s, 2H), 3.97 (d, J=12.3 Hz, 2H), 7.39-7.44 (dd, J=7.8 and 8.1 Hz, 1H), 7.52 (d, J=8.1 Hz, 1H), 7.67-7.75 (m, 2H), 7.87 (d, J=8.1 Hz, 1H), 7.96 (s, 1H), 8.21 (d, J=8.1 Hz, 1H), 9.57 (s, 1H), 10.15 (s, 1H), 12.25 (s, 1H). MS (ES⁺): m/z 403.98 (M+1).

### t) N-[5,6-Dihydro-5-oxo-indeno[1,2-c]isoquinolin-9-yl]-2-hydroxy-acetamide (59)

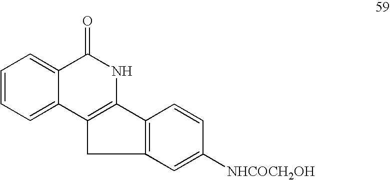

To a suspension of 5,6-dihydro-5-oxo-9-amino-indeno[1,2-c]isoquinoline (Compound 54a) (0.5 g, 0.002 mol) in saturated NaHCO₃ (100 ml) and ethyl acetate (100 ml) was added chloroacetoxy acetyl chloride (10 eq). Then the reaction mixture was stirred at room temperature for 1 h. The resultant solid was isolated by filtration and washed with ethyl acetate, water and finally with methanol. The solid was dried under vacuum to provide an acetoxy intermediate (0.560 g, 80%). The acetoxy intermediate (0.5 g, 0.0014 mol) was suspended in ethanol (50 ml) and treated with the hydrazine monohydrate (10 ml, excess) at room temperature and the reaction mixture was refluxed overnight. The resultant solid was filtered and washed with water and ethanol and dried under vacuum to provide Compound 59 (0.360 g, 82%). ¹H-NMR (DMSO-d₆): δ, 3.89 (s, 2H), 4.02 (s, 2H), 5.70 (s, 1H), 7.41-7.46 (dd, J=6.6 and 6.9 Hz, 1H), 7.65-7.77 (m, 3H), 7.90 (d, J=8.1 Hz, 1H), 8.08 (S, 1H), 8.23 (D, J=7.8 HZ, 1H), 9.78 (S, 1H), 12.26 (S, 1H); MS (ES⁺): m/z 307.1 (M+1).

### u) 1-Ethyl-3-(5-oxo-5,11-dihydro-6H-indeno[1,2-c]isoquinolin-9-yl)-urea (60)

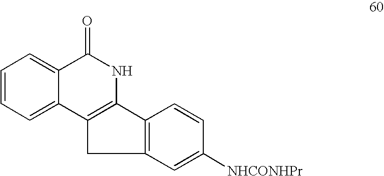

To a suspension of 5,6-dihydro-5-oxo-9-amino-indeno[1,2-c]isoquinoline (Compound 54a) (25 mg, 0.1 mmol) in DMF (3 ml) was added n-propylisocyanate (0.5 ml, excess). The reaction mixture was stirred at 110° C. for 6 h. The resultant solid was isolated by filtration and washed with methanol, water and finally with methanol. The resultant solid was dried under vacuum to provide Compound 60 (14 mg, 42%). ¹H NMR (DMSO-D₆): δ 0.84-0.89 (t, J=7.5 Hz, 4H), 1.40-1.47 (m, 2H), 3.01-3.07 (m, 2H), 3.82 (s, 2H), 6.17-6.20 (m, 1H), 7.25-7.28 (dd, J=8.1 and 1.5 Hz, 1H), 7.36-7.41 (dd, J=6.6 and 6.9 Hz, 1H), 7.66-7.72 (m, 2H), 7.78-7.81 (m, 2H), 8.20 (d, J=7.2 Hz, 1H), 8.55 (s, 1H), 12.21 (s, 1H); MS (ES⁺): m/z 334.3 (M+1).

### v) Effect of Illustrative Isoquinoline Compounds on In Vitro PARS Activity

The ability of an illustrative Isoquinoline Compound to inhibit PARS and prevent peroxynitrite-induced cytotoxicity can be demonstrated using methods described in Virag et al., Br. J. Pharmacol. 1999, 126(3):769-77; and Immunology 1998, 94(3):345-55.

The potency of inhibition on purified PARS enzyme can be subsequently determined for selected Isoquinoline Compounds, and the potency is compared with that of 3-aminobenzamide, a prototypical benchmark PARS inhibitor. The assay is performed in 96 well ELISA plates according to instructions provided with a commercially available PARS inhibition assay kit (for example, from Trevigen, Gaithersburg, Md.).

### w) Effect of Illustrative Isoquinoline Compounds on PARS Activity Using Cell Protection Assay

The ability of illustrative Isoquinoline Compounds to inhibit PARS and prevent peroxynitrite induced cytotoxicity was measured in a cell protection assay using the methods described in Jagtap et al., Bioorg. & Med. Chem. Letters 14 (2004) 81-85. Briefly, raw mouse macrophages were cultured then treated with an illustrative Isoquinoline Compound at various concentrations ranging from 10 nM to 10 μM for about 15 minutes. Peroxynitrite (750 μM) was then added to the treated macrophages for a 15 minute incubation period to induce PARS activation. The media was removed and replaced with 0.5 mL HEPES (pH 7.5) containing 0.01% digitonin and ³H-NAD (0.5 μCi/mL, final concentration of NAD+ in buffer is 20 nM/L) and the resultant mixture was allowed to stand for 20 minutes. The cells were then scraped from the wells and placed in Eppendorf tubes containing 50% (w/v) of ice-cold TCA (200 μL). The tubes were maintained at 4° C. for four hours, centrifuged at 1800 g for 10 minutes, and the supernatant removed. The resultant pellets were washed with 5% (w/v) TCA (200 μL, 2×), then solubilized overnight in 2% (w/v) SDS/0.1 N NaOH (250 μL) at 37° C. The contents of the tubes were then added to ScintiSafe Plus scintillation liquid (6.5 ml, Fisher Scientific) and radioactivity was measured using a liquid scintillation counter (Wallac, Gaithersburg, Md.). The results shown in Table 1 demonstrate that the illustrative Isoquinoline Compounds dose-dependently inhibit the activation of PARS.

### x) Effects of Isoquinoline Compounds in an In Vitro Model of Cell Death

Using an in vitro, oxidant-stimulated thymocyte assay (described, in detail, in Virag et al., Immunology 94(3):345-55, 1998), an illustrative Isoquinoline Compound can be tested for its ability to prevent oxidant-induced suppression of the viability of the cells. As such, this assay represents an in vitro model of reperfusion related cell death in ischemic organs.

### y) Effect of Isoquinoline Compounds on In Vivo Models of Inflammatory Diseases

In order to substantiate the efficacy of the compounds in inflammatory diseases, the effect of an illustrative Isoquinoline Compound can be determined usng a systemic inflammatory model induced by bacterial lipopolysaccharide (LPS), which is reported to be responsible for causing reperfusion injurys and inflammatory diseases such as septic shock and systemic inflammatory response syndrome in animals (see Parrillo, N. Engl. J. Med., 328:1471-1478 (1993) and Lamping, J. Clin. Invest. 101:2065-2071 (1998).

### z) Determination of the Effect of Isoquinoline Compounds on In Vivo Models of Reperfusion Injury

The efficacy of an illustrative Isoquinoline Compound in a mouse model of ischemic and reperfused gut can be determined according to the method described in Liaudet et al., Shock 2000, 14(2):134-41.

In another set of experiments, the effect of an illustrative Isoquinoline Compound in a rat model of middle cerebral artery occlusion/reperfusion can be assayed as described in Abdelkarim et al., Int J Mol Med. 2001, 7(3):255-60.

### aa) Effect of Illustrative Isoquinoline Compounds in an In Vivo Model of Diabetes

PARS inhibitors and PARS deficiency are known to reduce the development of diabetes and the incidence of diabetic complications. In order to substantiate the efficacy of an illustrative Isoquinoline Compound in a diabetes model, a single high-dose streptozotocin model of diabetes can be used as conducted as described in Mabley et al., Br J Pharmacol. 2001, 133(6):909-9; and Soriano et al., Nat Med. 2001, 7(1):108-13. Briefly, 160 mg/kg streptozotocin is injected to mice treated with vehicle (control) or with an illustrative Isoquinoline Compound intraperitoneally (3 mg/kg) and 3 days later blood sugar levels are determined using a blood glucose meter.

### bb) PARS Inhibitory Activity of Indeno[1,2-c]isoquinolinones: Purified PARS Enzyme Assay

The potency of inhibition on purified PARS enzyme was subsequently determined for illustrative compounds, and the potency was compared with that of 3-aminobenzamide, a prototypical benchmark PARS inhibitor. The assay was performed in 96 well ELISA plates according to instructions provided with a commercially available PARS inhibition assay kit (Trevigen, Gaithersburg, Md.). Briefly, wells were coated with 1 mg/ml histone (50 μl/well) at 4 C overnight. Plates were then washed four times with PBS and then blocked by adding 50 μl Strep-Diluent (supplied with the kit). After incubation (1 h, 25° C.), the plates were washed four times with PBS. Appropriate dilutions of compound was combined with 2× PARS cocktail (1.95 mM NAD+, 50 μM biotinylated NAD⁺ in 50 mM TRIS pH 8.0, 25 mM MgCl₂) and high specific activity PARS enzyme (both were supplied with the kit) in a volume of 50 μl. The reaction was allowed to proceed for 30 min at room temperature. After 4 washes in PBS, incorporated biotin was detected by peroxidase-conjugated streptavidin (1:500 dilution) and TACS Sapphire substrate. The assay confirmed the results of the macrophage-based PARS assay.

### cc) Cell Protection Assay

Raw murine macrophages were treated with Isoquinoline Compounds for 15 minutes prior to the addition of peroxynitrite (750 μM) for a further 15 minutes. For the measurement of PARS activity, the media were removed and replaced with 0.5 ml HEPES (pH 7.5) containing 0.01% digitonin and ³H-NAD (0.5 μCi ml⁻¹, final concentration of NAD⁺ in buffer is 20 nM/L) for 20 minutes. The cells were then scraped from the wells and placed in Eppendorf tubes containing 200 μl of 50% (w/v) ice-cold trichloroacetic acid (TCA). The tubes were then placed at 4° C. After 4 hr the tubes were centrifuged at 1800 g for 10 minutes and the supernatant removed. The pellets were washed twice with 500 μl ice-cold 5% TCA. The pellets were solubilized in 250 μl NaOH (0.1 M) containing 2% SDS overnight at 37° C. and the PARS activity was then determined by measuring the radioactivity incorporated using a Wallac scintillation counter. The solubilized protein (250 μl) was mixed with 5 ml of scintillant (ScintiSafe Plus, Fisher Scientific) before being counted for 10 minutes. The EC₅₀ value was calculated from a dose-response curve.

TABLE 1
Inhibitory effect of illustrative Isoquinoline Compounds on cell protection

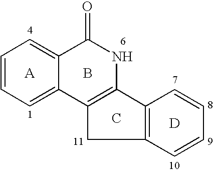

| | C₈ Position | C₉ Position | C₁₀ Position | IC₅₀ | EC₅₀ |
|---|---|---|---|---|---|
| 56a | NHCOCH₂NMe₂ | H | H | 0.8 | NT |
| 56b | NHCOCH₂morpholine | H | H | >3.0 | NT |
| 56d | NHCO(CH₂)₃morpholine | H | H | 0.4 | NT |
| 56c | NHCOCH₂(N-Me | H | H | >3.0 | NT |
| | piperazine) | | | | |
| 57 | H | NHCOCH₂NMe₂ | H | 0.1 | NT |
| 58 | H | NHCO(CH₂)₃morpholine | H | 0.1 | NT |
| 11a | H | NHCOCH₂[p-F-phenyl- | H | 0.07 | 0.08 |
| | | (piperazine)] | | | |
| 12a | H | NHCOCH₂[4-phenyl- | H | 0.08 | NT |
| | | (1,2,5,6- | | | |
| | | tetrahydropyridine)] | | | |
| 26 | H | NHCOCH₂[4-fluoro- | H | >0.3 | NT |
| | | phenyl-(1,2,5,6- | | | |
| | | tetrahydropyridine)] | | | |
| 27 | H | NHCONHpropyl | H | 0.1 | NT |
| 28 | H | NHCOCH₂OH | H | 0.11 | NT |
| 3a | H | H | NHCOCH₂NMe₂ | NT | 0.2 |
| 3b | H | H | NHCO(CH₂)₂N | NT | 0.28 |
| | | | Me₂ | | |

NT = Not tested

The present invention is not to be limited in scope by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited, the entire disclosures of which have been incorporated herein in their entirety.

What is claimed is:

1. A compound having the formula: 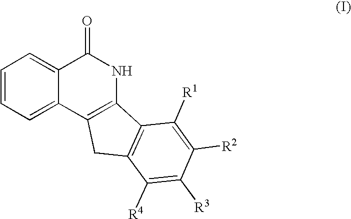
or a pharmaceutically acceptable salt thereof
wherein
R² and R³ are hydrogen;
one of the R¹ and R⁴ groups is —NHC(O)—(CH₂)ₙ—NR⁵R⁶ and the remaining group is hydrogen;
R⁵ and R⁶ are independently —H, —C₁-C₆ alkyl, -phenyl, or benzyl, wherein the —C₁-C₆ alkyl, -phenyl, or benzyl, is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₆ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; and
n is an integer ranging from 1 to 6.

2. The compound or pharmaceutically acceptable salt of the compound of claim 1, wherein R¹ is —NH(CH₂)ₙ—N(R⁵)(R⁶).

3. The compound or pharmaceutically acceptable salt of the compound of claim 1, wherein R⁴ is —NH(CH₂)ₙ—N(R⁵)(R⁶).

4. The compound or pharmaceutically acceptable salt of the compound of claim 1, wherein R⁵ is and R⁶ are each C₁-C₆ alkyl.

5. The compound or pharmaceutically acceptable salt of the compound of claim 1, wherein R⁵ is and R⁶ are each methyl.

6. The compound or pharmaceutically acceptable salt of the compound of claim 1, wherein n is 1.

7. The compound or pharmaceutically acceptable salt of the compound of claim 1, wherein n is 2.

8. The compound or pharmaceutically acceptable salt of the compound of claim 1, wherein n is 3.

9. The compound or pharmaceutically acceptable salt of the compound of claim 1, wherein —N(R⁵)(R⁶) is

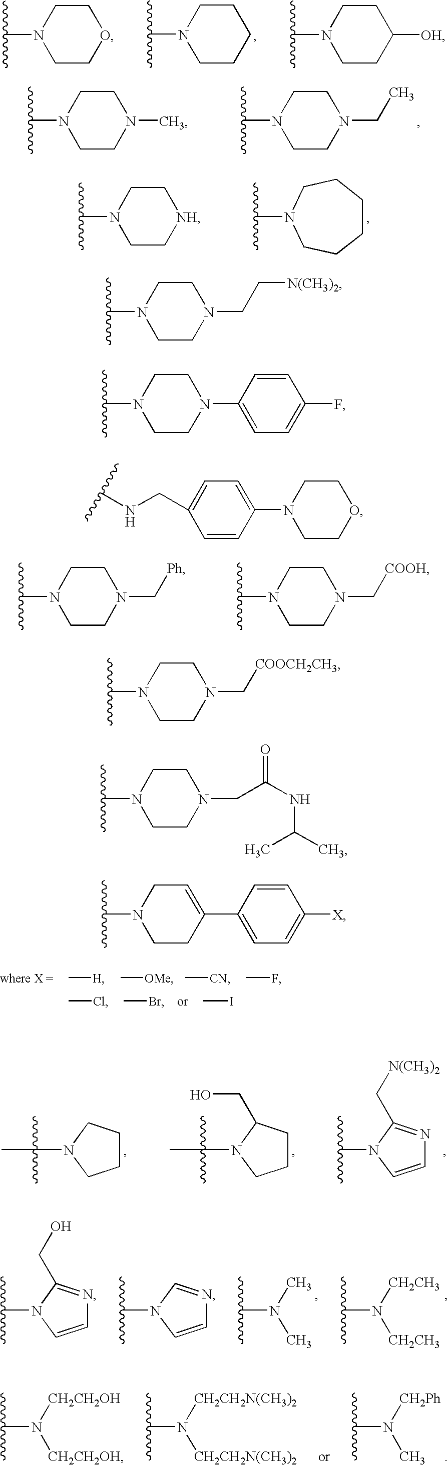

10. The compound or pharmaceutically acceptable salt of the compound of claim 1, wherein —N(R⁵)(R⁶) is —(N-morpholino).

11. A compound having the formula: 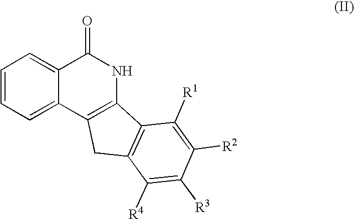
or a pharmaceutically acceptable salt thereof
wherein
one of the R¹, R², R³, and R⁴ groups is —NHC(O)—(CH₂)ₙ—NZ₁Z₂ and the remaining groups are simultaneously hydrogen;
one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₁ and Z₂ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; and
n is an integer ranging from 1 to 6.

12. The compound or pharmaceutically acceptable salt of the compound of claim 11, wherein R¹ is —NHC(O)(CH₂)ₙ—N(Z₁)(Z₂).

13. The compound or pharmaceutically acceptable salt of the compound of claim 11, wherein R² is —NHC(O)(CH₂)ₙ—N(Z₁)(Z₂).

14. The compound or pharmaceutically acceptable salt of the compound of claim 11, wherein R³ is —NHC(O)(CH₂)ₙ—N(Z₁)(Z₂).

15. The compound or pharmaceutically acceptable salt of the compound of claim 11, wherein R⁴ is —NHC(O)(CH₂)ₙ—N(Z₁)(Z₂).

16. The compound or pharmaceutically acceptable salt of the compound of claim 11, wherein n is 1.

17. The compound or pharmaceutically acceptable salt of the compound of claim 11, wherein n is 2.

18. The compound or pharmaceutically acceptable salt of the compound of claim 11, wherein n is 3.

19. The compound or pharmaceutically acceptable salt of the compound of claim 11, wherein —N(Z₁)(Z₂) is

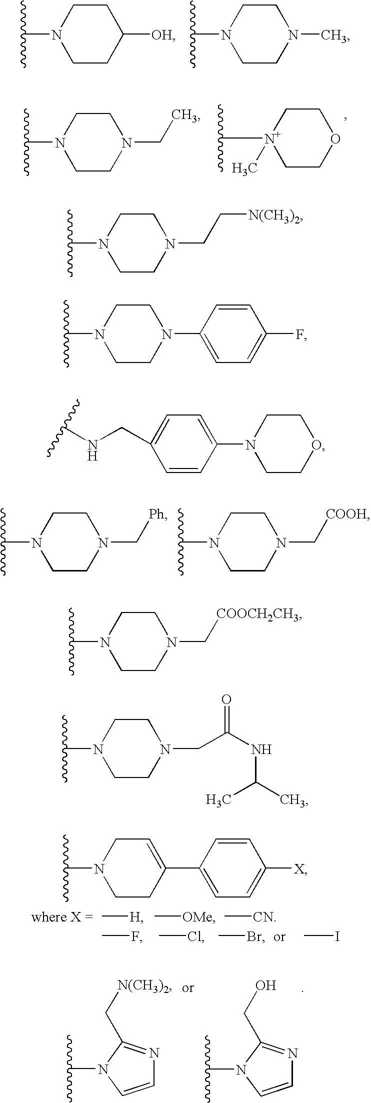

20. The compound or pharmaceutically acceptable salt of the compound of claim 11, wherein —N(Z₁)(Z₂) is

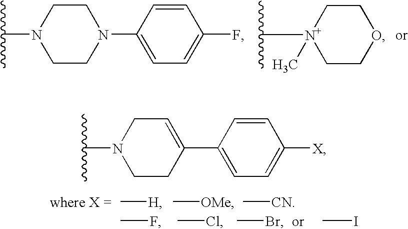

21. A compound having the formula: 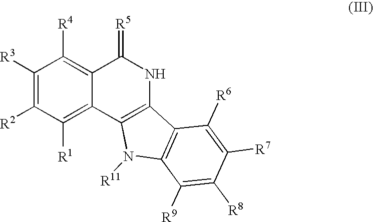
or a pharmaceutically acceptable salt thereof,
wherein:
R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂, -halo, —OH, —NH₂, or -A-B;
R⁵ is O, S or NH;
A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹¹ is —C(O)O—(C₁-C₅ alkylene)-NZ₅Z₆;
one of Z₅ and Z₆ is —H, —C₁-C₆ alkyl or -phenyl, and the other of Z₅ and Z₆ is phenyl, wherein the -phenyl in each instance is unsubstituted or substituted with one or more of -halo, —OH or —N(Z₇)(Z₈), where N, Z₇ and Z₈ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₅ and Z₆ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl;
each n is independently an integer ranging from 1 to 10; and
each p is independently an integer ranging from 0 to 5.

22. The compound or pharmaceutically acceptable salt of the compound of claim 21, wherein R⁵ is O.

23. The compound or pharmaceutically acceptable salt of the compound of claim 21, wherein R¹-R⁴ are each hydrogen.

24. The compound or pharmaceutically acceptable salt of the compound of claim 21, wherein R⁶-R⁹ are each hydrogen.

25. The compound or pharmaceutically acceptable salt of the compound of claim 21, wherein R¹¹ is —C(O)O—(C₁-C₅ alkylene)-NZ₅Z₆, where —N(Z₅)(Z₆) is:

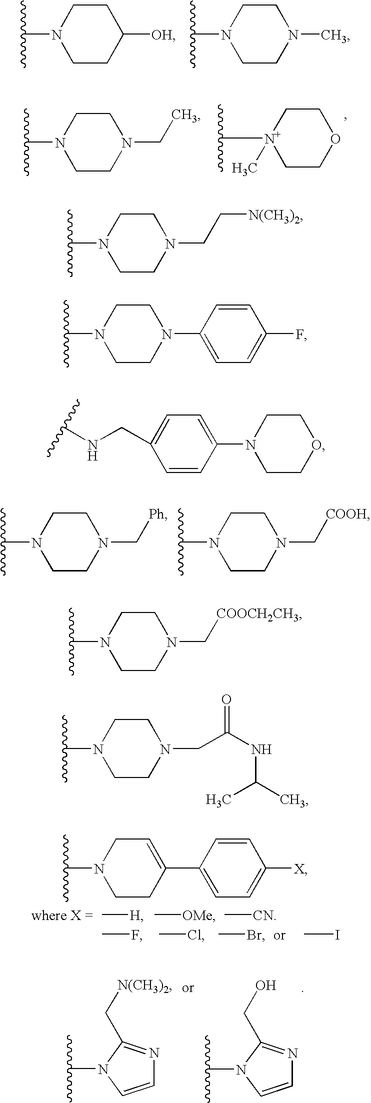

26. The compound or pharmaceutically acceptable salt of the compound of claim 21, wherein R¹¹ is —C(O)O—(C₁-C₅ alkylene)-NZ₅Z₆, where —N(Z₅)(Z₆) is:

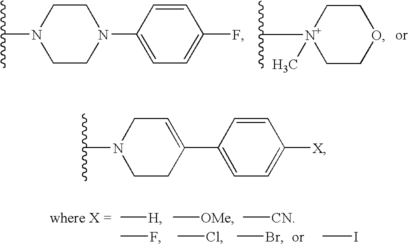

27. A compound having the formula 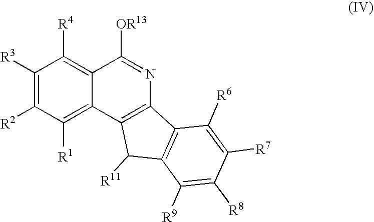
or a pharmaceutically acceptable salt thereof,
wherein:
R¹, R², R³, R⁴, R⁶, R⁷, R⁸ and R⁹ are each independently —H, —O—(C₁-C₅ alkyl), —C₁-C₁₀ alkyl, —C₂-C₁₀ alkenyl, -aryl, —C(O)OH, —C(O)O(C₁-C₅ alkyl), —OC(O)(C₁-C₅ alkyl), —NO₂, —NHC(O)(CH₂)ₙ—NH₂, —NHSO₂NH(CH₂)ₙ—NH₂, —C(O)NH(CH₂)ₙ—NH₂, —SO₂NH(CH₂)ₙ—NH₂, -halo, —OH, —NH₂, or -A-B;
A is —SO₂—, —SO₂NH—, —NHCO—, —NHCONH—, —O—, —CO—, —OC(O)—, —C(O)O—, —CONH—, —CON(C₁-C₅ alkyl)-, —NH—, —(CH₂)ₚ—, —S— or —C(S)—;
B is —C₁-C₁₀ alkyl, —C₂-C₁₀ alkenyl, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, (nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), -aryl, —NZ₁Z₂, —(C₁-C₅ alkylene)-NZ₁Z₂, —C(O)OH, —C(O)O—(C₁-C₅ alkyl), —C(O)O-aryl or —C(NH)NH₂, each of which other than —NZ₁Z₂, C(O)OH, or —C(NH)NH₂, is unsubstituted or substituted with one or more of —C(O)NH₂, —O—(C₁-C₅ alkyl), -halo, —OH, —NO₂, —NH₂, —CN, —C₁-C₁₀ alkyl, -aryl, —C(O)OH, or —C(O)O—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, —OH or —NH₂; or N, Z₃ and Z₄ are taken together to form a -(nitrogen-containing-3- to 7-membered monocyclic heterocycle) or a -(nitrogen-containing 7- to 10-membered bicyclic heterocycle), or N, Z₁ and Z₂ are taken together to form a -(nitrogen-containing-3- to 7-membered monocyclic heterocycle) or a -(nitrogen-containing 7- to 10-membered bicyclic heterocycle);
R¹¹ is —C(O)O—(C₁-C₅ alkylene)-NZ₅Z₆;
one of Z₅ and Z₆ is —H, —C₁-C₆ alkyl, or -phenyl, and the other of Z₅ and Z₆ is phenyl, wherein the -phenyl in each instance is unsubstituted or substituted with one or more of -halo, —OH or —N(Z₇)(Z₈), where N, Z₇ and Z₈ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl; or N, Z₅ and Z₆ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl;
R¹³ is —C₁-C₁₀ alkyl, —C(O)—C₁-C₁₀ alkyl, —C(O)-aryl, —C(O)-(3- to 7-membered monocyclic heterocycle), or -glycoside, each of which is unsubstituted or substituted with one or more -halo, —C(O)OH, or —OH groups;
each n is independently an integer ranging from 1 to 10; and
each p is independently an integer ranging from 0 to 5.

28. The compound or pharmaceutically acceptable salt of the compound of claim 27, wherein R¹-R⁴ are each hydrogen.

29. The compound or pharmaceutically acceptable salt of the compound of claim 27, wherein R⁶-R⁹ are each hydrogen.

30. The compound or pharmaceutically acceptable salt of the compound of claim 27, wherein R¹¹ is —C(O)O—(C₁-C₅ alkylene)-NZ₅Z₆, where —N(Z₅)(Z₆) is:

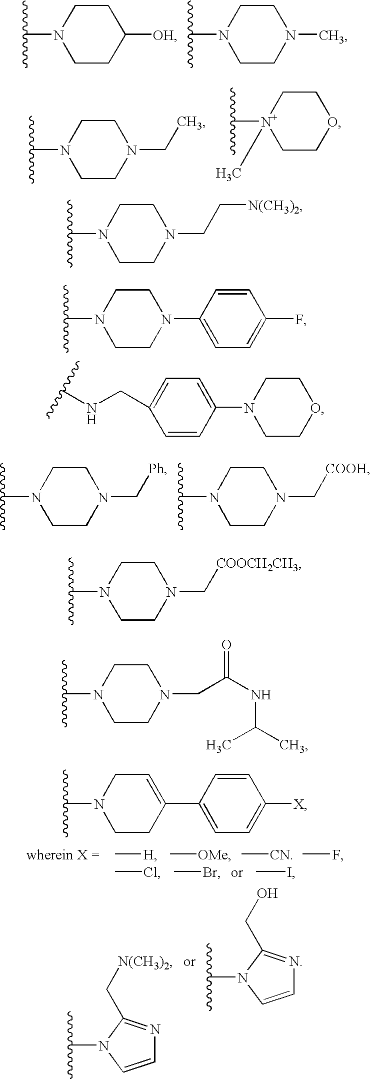

31. The compound or pharmaceutically acceptable salt of the compound of claim 27, wherein R¹¹ is —C(O)O—(C₁-C₅ alkylene)-NZ₅Z₆, where —N(Z₅)(Z₆) is:

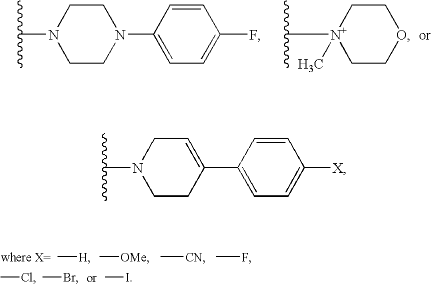

32. A composition comprising a physiologically acceptable carrier or vehicle and an effective amount of a compound or a pharmaceutically acceptable salt of a compound of claim 1.

33. A composition comprising a physiologically acceptable carrier or vehicle and an effective amount of a compound or a pharmaceutically acceptable salt of a compound of claim 11.

34. A composition comprising a physiologically acceptable carrier or vehicle and an effective amount of a compound or a pharmaceutically acceptable salt of a compound of claim 21.

35. A composition comprising a physiologically acceptable carrier or vehicle and an effective amount of a compound or a pharmaceutically acceptable salt of a compound of claim 27.

36. A composition comprising a physiologically acceptable carrier or vehicle, an effective amount of a compound or a pharmaceutically acceptable salt of a compound of claim 1, and an effective amount of temozolomide.

37. A composition comprising a physiologically acceptable carrier or vehicle, an effective amount of a compound or a pharmaceutically acceptable salt of a compound of claim 11, and an effective amount of temozolomide.

38. A composition comprising a physiologically acceptable carrier or vehicle, an effective amount of a compound or a pharmaceutically acceptable salt of a compound of claim 21, and an effective amount of temozolomide.

39. A composition comprising a physiologically acceptable carrier or vehicle, an effective amount of a compound or a pharmaceutically acceptable salt of a compound of claim 27, and an effective amount of temozolomide.

40. A composition comprising a physiologically acceptable carrier or vehicle, an effective amount of a compound or a pharmaceutically acceptable salt of a compound of claim 1, and an effective amount of procarbazine.

41. A composition comprising a physiologically acceptable carrier or vehicle, an effective amount of a compound or a pharmaceutically acceptable salt of a compound of claim 11, and an effective amount of procarbazine.

42. A composition comprising a physiologically acceptable carrier or vehicle, an effective amount of a compound or a pharmaceutically acceptable salt of a compound of claim 21, and an effective amount of procarbazine.

43. A composition comprising a physiologically acceptable carrier or vehicle, an effective amount of a compound or a pharmaceutically acceptable salt of a compound of claim 27, and an effective amount of procarbazine.

44. A composition comprising a physiologically acceptable carrier or vehicle, an effective amount of a compound or a pharmaceutically acceptable salt of a compound of claim 1, and an effective amount of dacarbazine.

45. A composition comprising a physiologically acceptable carrier or vehicle, an effective amount of a compound or a pharmaceutically acceptable salt of a compound of claim 11, and an effective amount of dacarbazine.

46. A composition comprising a physiologically acceptable carrier or vehicle, an effective amount of a compound or a pharmaceutically acceptable salt of a compound of claim 21, and an effective amount of dacarbazine.

47. A composition comprising a physiologically acceptable carrier or vehicle, an effective amount of a compound or a pharmaceutically acceptable salt of a compound of claim 27, and an effective amount of dacarbazine.

48. A composition comprising a physiologically acceptable carrier or vehicle, an effective amount of a compound or a pharmaceutically acceptable salt of a compound of claim 1, and an effective amount of irinotecan.

49. A composition comprising a physiologically acceptable carrier or vehicle, an effective amount of a compound or a pharmaceutically acceptable salt of a compound of claim 11, and an effective amount of irinotecan.

50. A composition comprising a physiologically acceptable carrier or vehicle, an effective amount of a compound or a pharmaceutically acceptable salt of a compound of claim 21, and an effective amount of irinotecan.

51. A composition comprising a physiologically acceptable carrier or vehicle, an effective amount of a compound or a pharmaceutically acceptable salt of a compound of claim 27, and an effective amount of irinotecan.

52. A composition comprising a physiologically acceptable carrier or vehicle, an effective amount of a compound or a pharmaceutically acceptable salt of a compound of claim 1, and an effective amount of Interleukin-2.

53. A composition comprising a physiologically acceptable carrier or vehicle, an effective amount of a compound or a pharmaceutically acceptable salt of a compound of claim 11, and an effective amount of Interleukin-2.

54. A composition comprising a physiologically acceptable carrier or vehicle, an effective amount of a compound or a pharmaceutically acceptable salt of a compound of claim 21, and an effective amount of Interleukin-2.

55. A composition comprising a physiologically acceptable carrier or vehicle, an effective amount of a compound or a pharmaceutically acceptable salt of a compound of claim 27, and an effective amount of Interleukin-2.

56. A method for treating cancer, comprising administering an effective amount of a compound of claim 1 or a pharmaceutically acceptable salt thereof to a subject in need thereof.

57. A method for treating cancer, comprising administering an effective amount of a compound of claim 11 or a pharmaceutically acceptable salt thereof to a subject in need thereof.

58. A method for treating cancer, comprising administering an effective amount of a compound of claim 21 or a pharmaceutically acceptable salt thereof to a subject in need thereof.

59. A method for treating cancer, comprising administering an effective amount of a compound of claim 27 or a pharmaceutically acceptable salt thereof to a subject in need thereof.

60. The method of claim 56, wherein the cancer is lung cancer, breast cancer, colorectal cancer, prostate cancer, testicular cancer, leukemia, lymphoma, non-Hodgkin's lymphoma, skin cancer, brain cancer, a cancer of the central nervous system, ovarian cancer, uterine cancer, cervical cancer, stomach cancer, pancreatic cancer, esophageal cancer, kidney cancer, liver cancer, or a head and neck cancer.

61. The method of claim 57, wherein the cancer is lung cancer, breast cancer, colorectal cancer, prostate cancer, testicular cancer, leukemia, lymphoma, non-Hodgkin's lymphoma, skin cancer, brain cancer, a cancer of the central nervous system, ovarian cancer, uterine cancer, cervical cancer, stomach cancer, pancreatic cancer, esophageal cancer, kidney cancer, liver cancer, or a head and neck cancer.

62. The method of claim 57, wherein the cancer is lung cancer, breast cancer, colorectal cancer, prostate cancer, testicular cancer, leukemia, lymphoma, non-Hodgkin's lymphoma, skin cancer, brain cancer, a cancer of the central nervous system, ovarian cancer, uterine cancer, cervical cancer, stomach cancer, pancreatic cancer, esophageal cancer, kidney cancer, liver cancer, or a head and neck cancer.

63. The method of claim 59, wherein the cancer is lung cancer, breast cancer, colorectal cancer, prostate cancer, testicular cancer, leukemia, lymphoma, non-Hodgkin's lymphoma, skin cancer, brain cancer, a cancer of the central nervous system, ovarian cancer, uterine cancer, cervical cancer, stomach cancer, pancreatic cancer, esophageal cancer, kidney cancer, liver cancer, or a head and neck cancer.

64. The method of claim 56, further comprising administering an effective amount of another anticancer agent.

65. The method of claim 57, further comprising administering an effective amount of another anticancer agent.

66. The method of claim 58, further comprising administering an effective amount of another anticancer agent.

67. The method of claim 59, further comprising administering an effective amount of another anticancer agent.

68. The method of claim 64, wherein the other anticancer agent is temozolomide, procarbazine, dacarbazine, irinotecan, Interleukin-2, or a combination thereof.

69. The method of claim 65, wherein the other anticancer agent is temozolomide, procarbazine, dacarbazine, irinotecan, Interleukin-2, or a combination thereof.

70. The method of claim 66, wherein the other anticancer agent is temozolomide, procarbazine, dacarbazine, irinotecan, Interleukin-2, or a combination thereof.

71. The method of claim 67, wherein the other anticancer agent is temozolomide, procarbazine, dacarbazine, irinotecan, Interleukin-2, or a combination thereof.

72. The method of claim 56, wherein the cancer is metastatic brain cancer, glioma, or melanoma.

73. The method of claim 57, wherein the cancer is metastatic brain cancer, glioma, or melanoma.

74. The method of claim 58, wherein the cancer is metastatic brain cancer, glioma, or melanoma.

75. The method of claim 59, wherein the cancer is metastatic brain cancer, glioma, or melanoma.

76. The method of claim 72, wherein the glioma is pilocytic astrocytoma, astrocytoma, anaplastic astrocytoma, or glioblastoma multiforme.

77. The method of claim 73, wherein the glioma is pilocytic astrocytoma, astrocytoma, anaplastic astrocytoma, or glioblastoma multiforme.

78. The method of claim 74, wherein the glioma is pilocytic astrocytoma, astrocytoma, anaplastic astrocytoma, or glioblastoma multiforme.

79. The method of claim 75, wherein the glioma is pilocytic astrocytoma, astrocytoma, anaplastic astrocytoma, or glioblastoma multiforme.

80. A method for treating renal failure, comprising administering an effective amount of a compound of claim 1 or a pharmaceutically acceptable salt thereof to a subject in need thereof.

81. A method for treating renal failure, comprising administering an effective amount of a compound of claim 11 or a pharmaceutically acceptable salt thereof to a subject in need thereof.

82. A method for treating renal failure, comprising administering an effective amount of a compound of claim 21 or a pharmaceutically acceptable salt thereof to a subject in need thereof.

83. A method for treating renal failure, comprising administering an effective amount of a compound of claim 27 or a pharmaceutically acceptable salt thereof to a subject in need thereof.

84. The method of claim 80, wherein the renal failure is chronic renal failure or acute renal failure.

85. The method of claim 81, wherein the renal failure is chronic renal failure or acute renal failure.

86. The method of claim 82, wherein the renal failure is chronic renal failure or acute renal failure.

87. The method of claim 83, wherein the renal failure is chronic renal failure or acute renal failure.

88. A method for treating a reperfusion injury, comprising administering an effective amount of a compound of claim 1 or a pharmaceutically acceptable salt thereof to a subject in need thereof.

89. A method for treating a reperfusion injury, comprising administering an effective amount of a compound of claim 11 or a pharmaceutically acceptable salt thereof to a subject in need thereof.

90. A method for treating a reperfusion injury, comprising administering an effective amount of a compound of claim 21 or a pharmaceutically acceptable salt thereof to a subject in need thereof.

91. A method for treating a reperfusion injury, comprising administering an effective amount of a compound of claim 27 or a pharmaceutically acceptable salt thereof to a subject in need thereof.

92. The method of claim 88, wherein the reperfusion injury is stroke or myocardial infarction.

93. The method of claim 89, wherein the reperfusion injury is stroke or myocardial infarction.

94. The method of claim 90, wherein the reperfusion injury is stroke or myocardial infarction.

95. The method of claim 91, wherein the reperfusion injury is stroke or myocardial infarction.

96. A method for treating an inflammatory disease, comprising administering an effective amount of a compound of claim 1 or a pharmaceutically acceptable salt thereof to a subject in need thereof.

97. A method for treating an inflammatory disease, comprising administering an effective amount of a compound of claim 11 or a pharmaceutically acceptable salt thereof to a subject in need thereof.

98. A method for treating an inflammatory disease, comprising administering an effective amount of a compound of claim 21 or a pharmaceutically acceptable salt thereof to a subject in need thereof.

99. A method for treating an inflammatory disease, comprising administering an effective amount of a compound of claim 27 or a pharmaceutically acceptable salt thereof to a subject in need thereof.

100. The method of claim 96, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

101. The method of claim 97, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

102. The method of claim 98, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

103. The method of claim 99, wherein the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, hemorrhagic shock, anaphylactic shock, traumatic shock or chemotherapeutic shock.

104. A method for treating diabetes, comprising administering an effective amount of a compound of claim 1 or a pharmaceutically acceptable salt thereof to a subject in need thereof.

105. A method for treating diabetes, comprising administering an effective amount of a compound of claim 11 or a pharmaceutically acceptable salt thereof to a subject in need thereof.

106. A method for treating diabetes, comprising administering an effective amount of a compound of claim 21 or a pharmaceutically acceptable salt thereof to a subject in need thereof.

107. A method for treating diabetes, comprising administering an effective amount of a compound of claim 27 or a pharmaceutically acceptable salt thereof to a subject in need thereof.

108. The method of claim 104, wherein the diabetes is type I diabetes or type II diabetes.

109. The method of claim 105, wherein the diabetes is type I diabetes or type II diabetes.

110. The method of claim 106, wherein the diabetes is type I diabetes or type II diabetes.

111. The method of claim 107, wherein the diabetes is type I diabetes or type II diabetes.

112. A method for treating an ischemic condition, comprising administering an effective amount of a compound of claim 1 or a pharmaceutically acceptable salt thereof to a subject in need thereof.

113. A method for treating an ischemic condition, comprising administering an effective amount of a compound of claim 11 or a pharmaceutically acceptable salt thereof to a subject in need thereof.

114. A method for treating an ischemic condition, comprising administering an effective amount of a compound of claim 21 or a pharmaceutically acceptable salt thereof to a subject in need thereof.

115. A method for treating an ischemic condition, comprising administering an effective amount of a compound of claim 27 or a pharmaceutically acceptable salt thereof to a subject in need thereof.

116. The method of claim 112, wherein the ischemic condition is myocardial ischemia, stable angina, unstable angina, stroke, ischemic heart disease or cerebral ischemia.

117. The method of claim 113, wherein the ischemic condition is myocardial ischemia, stable angina, unstable angina, stroke, ischemic heart disease or cerebral ischemia.

118. The method of claim 114, wherein the ischemic condition is myocardial ischemia, stable angina, unstable angina, stroke, ischemic heart disease or cerebral ischemia.

119. The method of claim 115, wherein the ischemic condition is myocardial ischemia, stable angina, unstable angina, stroke, ischemic heart disease or cerebral ischemia.

120. A method for treating reoxygenation injury resulting from organ transplantation, comprising administering an effective amount of a compound of claim 1 or a pharmaceutically acceptable salt thereof to a subject in need thereof.

121. A method for treating reoxygenation injury resulting from organ transplantation, comprising administering an effective amount of a compound of claim 11 or a pharmaceutically acceptable salt thereof to a subject in need thereof.

122. A method for treating reoxygenation injury resulting from organ transplantation, comprising administering an effective amount of a compound of claim 21 or a pharmaceutically acceptable salt thereof to a subject in need thereof.

123. A method for treating reoxygenation injury resulting from organ transplantation, comprising administering an effective amount of a compound of claim 27 or a pharmaceutically acceptable salt thereof to a subject in need thereof.

124. A method for treating Parkinson's disease, comprising administering an effective amount of a compound of claim 1 or a pharmaceutically acceptable salt thereof to a subject in need thereof.

125. A method for treating Parkinson's disease, comprising administering an effective amount of a compound of claim 11 or a pharmaceutically acceptable salt thereof to a subject in need thereof.

126. A method for treating Parkinson's disease, comprising administering an effective amount of a compound of claim 21 or a pharmaceutically acceptable salt thereof to a subject in need thereof.

127. A method for treating Parkinson's disease, comprising administering an effective amount of a compound of claim 27 or a pharmaceutically acceptable salt thereof to a subject in need thereof.

128. A method for treating a vascular disease, comprising administering an effective amount of a compound of claim 1 or a pharmaceutically acceptable salt thereof to a subject in need thereof.

129. A method for treating a vascular disease, comprising administering an effective amount of a compound of claim 11 or a pharmaceutically acceptable salt thereof to a subject in need thereof.

130. A method for treating a vascular disease, comprising administering an effective amount of a compound of claim 21 or a pharmaceutically acceptable salt thereof to a subject in need thereof.

131. A method for treating a vascular disease, comprising administering an effective amount of a compound of claim 27 or a pharmaceutically acceptable salt thereof to a subject in need thereof.

132. The method of claim 128, wherein the vascular disease is peripheral arterial occlusion, thromboangitis obliterans, Reynaud's disease and phenomenon, acrocyanosis, erythromelalgia, venous thrombosis, varicose veins, arteriovenous fistula, lymphedema or lipedema.

133. The method of claim 129, wherein the vascular disease is peripheral arterial occlusion, thromboangitis obliterans, Reynaud's disease and phenomenon, acrocyanosis, erythromelalgia, venous thrombosis, varicose veins, arteriovenous fistula, lymphedema or lipedema.

134. The method of claim 130, wherein the vascular disease is peripheral arterial occlusion, thromboangitis obliterans, Reynaud's disease and phenomenon, acrocyanosis, erythromelalgia, venous thrombosis, varicose veins, arteriovenous fistula, lymphedema or lipedema.

135. The method of claim 131, wherein the vascular disease is peripheral arterial occlusion, thromboangitis obliterans, Reynaud's disease and phenomenon, acrocyanosis, erythromelalgia, venous thrombosis, varicose veins, arteriovenous fistula, lymphedema or lipedema.

136. A method for treating a diabetic complication, comprising administering an effective amount of a compound of claim 1 or a pharmaceutically acceptable salt thereof to a subject in need thereof.

137. A method for treating a diabetic complication, comprising administering an effective amount of a compound of claim 11 or a pharmaceutically acceptable salt thereof to a subject in need thereof.

138. A method for treating a diabetic complication, comprising administering an effective amount of a compound of claim 21 or a pharmaceutically acceptable salt thereof to a subject in need thereof.

139. A method for treating a diabetic complication, comprising administering an effective amount of a compound of claim 27 or a pharmaceutically acceptable salt thereof to a subject in need thereof.

140. The method of claim 128, wherein the vascular disease is a cardiovascular disease.

141. The method of calim 129, wherein the vascular disease is a cardiovascular disease.

142. The method of claim 130, wherein the vascular disease is a cardiovascular disease.

143. The method of claim 131, wherein the vascular disease is a cardiovascular disease.

144. The method of claim 140, wherein the cardiovascular disease is chronic heart failure, atrial fibrillation, supraventricular tachycardia, atrial flutter or paroxysmal atrial tachycardia.

145. The method of claim 141, wherein the cardiovascular disease is chronic heart failure, atrial fibrillation, supraventricular tachycardia, atrial flutter or paroxysmal atrial tachycardia.

146. The method of claim 142, wherein the cardiovascular disease is chronic heart failure, atrial fibrillation, supraventricular tachycardia, atrial flutter or paroxysmal atrial tachycardia.

147. The method of claim 143, wherein the cardiovascular disease is chronic heart failure, atrial fibrillation, supraventricular tachycardia, atrial flutter or paroxysmal atrial tachycardia.

148. A compound having the formula: 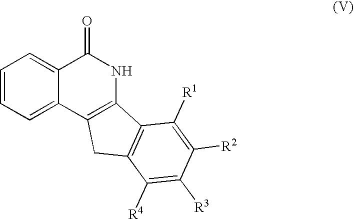
and pharmaceutically acceptable salts thereof
wherein
R³ is —NHC(O)—(CH₂)ₙ—X and R₁, R₂, R₄ are simultaneously hydrogen;
X is —OH, hydroxy-substituted C₁-C₆ alkyl, or NZ₁Z₂;
one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or -C₁-C₁₀ alkyl; or N, Z₁ and Z₂ are taken together to form a nitrogen-containing 3- to 7-membered monocyclic heterocycle which is unsubstituted or substituted with one to three of —C₁-C₅ alkyl, phenyl, benzyl, hydroxy-substituted C₁-C₅ alkyl, -halo, -halo-substituted C₁-C₅ alkyl, halo-substituted phenyl, hydroxy, —O—C₁-C₅ alkyl, —(O—C₁-C₅-alkyl)-substituted phenyl, cyano-substituted phenyl, —N(R^{a})₂, —(C₁-C₅ alkylene)-N(R^{a})₂, —COOH, —(C₁-C₅ alkylene)-COOH, —(C₁-C₅ alkylene)-C(O)O—C₁-C₅ alkyl, —(C₁-C₅-alkylene)-C(O)NH—C₁-C₅ alkyl, —C(O)O—(C₁-C₅ alkyl), —OC(O)—(C₁-C₅ alkyl), —C(O)NH₂, or —NO₂, wherein each occurrence of R^{a} is independently —H, -benzyl, or -C₁-C₁₀ alkyl; and
n is 0 or 1.

149. The compound or pharmaceutically acceptable salt of the compound of claim 148, wherein R³ is —NHC(O)—(CH₂)ₙ—N(Z₁)(Z₂) and R¹, R² and R⁴ are each hydrogen.

150. The compound or pharmaceutically acceptable salt of the compound of claim 148, wherein R³ is —NHC(O)—(CH₂)ₙ—OH and R¹, R² and R⁴ are each hydrogen.

151. The compound or pharmaceutically acceptable salt of the compound of claim 149, wherein Z₁ is H and Z₂ is propyl.
